(19) United States
(12) Patent Application Publication
(10) Pub. No.: US 2023/0265531 A1
(43) Pub. Date: Aug. 24, 2023

(54) COMPOSITIONS AND METHODS FOR ONE-STEP NUCLEIC ACID AMPLIFICATION AND DIAGNOSTIC METHODS BASED THEREON

(71) Applicant: King Abdullah University of Science and Technology, Thuwal (SA)

(72) Inventors: Samir M. Hamdan, Thuwal (SA); Masateru Takahashi, Thuwal (SA); Muhammad Tehseen, Thuwal (SA); Etsuko Takahashi, Thuwal (SA)

(21) Appl. No.: 18/006,420

(22) PCT Filed: Jul. 22, 2021
(86) PCT No.: PCT/IB2021/056646

§ 371 Date: Jan. 23, 2023

Related U.S. Application Data
(60) Provisional application No. 63/055,250, filed on Jul. 22, 2020.
(60) Provisional application No. 63/214,738, filed on Jun. 24, 2021.

(51) Int. Cl. C12Q 1/70; C12Q 1/6851

(52) U.S. Cl. CPC C12Q 1/701; C12Q 1/6851

(57) ABSTRACT

Compositions and methods for a nucleic acid amplification and diagnostic methods based therein, are disclosed. The compositions include purified Thermus aquaticus DNA polymerase (Taq Pol) and Moloney Murine Leukemia Virus Reverse Transcriptase (MMLV-RT), in proportions and under reaction conditions that allow one-step amplification of a nucleic acid of interest. In particular, the compositions are useful in one-step RT-qPCR and RT-LAMP methods. The method includes sampling the specimen from a subject, extracting and purifying RNA from the sample. The compositions and methods may be used to produce, analyze, quantitate, detect and otherwise manipulate nucleic acid molecules. For example, disclosed are methods of detecting the presence of a viral nucleic acid in a sample from a subject. Methods of diagnosing a subject as being infected with a viral pathogen are also provided. A preferred virus or pathogen is SARS-CoV-2.

### CROSS REFERENCE TO RELATED APPLICATIONS

This application claims the benefit of and priority to U.S. Provisional Application No. 63/055,250, filed Jul. 22, 2020, and U.S. Provisional Application No. 63/214,738, filed Jun. 24, 2021, which are hereby incorporated herein by reference in their entirety.

### FIELD OF THE INVENTION

This invention is generally in the field of compositions and methods for one-step detection of pathogens in a sample.

### BACKGROUND OF THE INVENTION

In December 2019, an outbreak of a new syndrome characterized by serious symptoms including fever, severe respiratory illness, and acute pneumonia eventually leading to respiratory failure and death was reported in Wuhan city of Hubei province in China. This disease swiftly spread out across the globe resulting in unprecedented preventive measures worldwide [1, 2]. On Jan. 7, 2020, the Chinese health authorities confirmed that this recently discovered syndrome was associated with a new member of the coronavirus (CoV) family closely related to a group of severe acute respiratory syndrome coronaviruses (SARS-CoV) [3, 4]. On February 11th, the World Health Organization (WHO) designated the name “coronavirus disease 2019” abbreviated as (COVID-19) to this highly contagious disease [5]. As of July 11th, there has been nearly 12.6 million confirmed COVID-19 cases including over 562,000 fatalities globally [6].

The COVID-19 pandemic is caused by the new strain (SARS-CoV-2) classified under genus Betacoronavirus (0-CoV) and subgenus Sarbecovirus [7, 8]. Large numbers of SARS-related coronaviruses (SARSr-CoVs) have been discovered in bats, which are their natural hosts [9-11]. SARS-CoV-2 is 96% identical at the whole genome level to bat CoV and shares 79.6% sequence identity to SARS-CoV [7]. Coronaviruses are characterized by large, single-stranded (ss), positive-sense RNA genomes ranging from 26 to 32 kilo bases (kb) [12]. Coronaviruses express their replication and transcription complexes, including RNA-dependent RNA polymerase (RdRp), from a single large open reading frame referred to as ORFIab [13]. The viral particle contains four main structural proteins: Spike (S), Membrane (M), Envelope (E), and Nucleocapsid (N) proteins [14].

The S gene in CoVs codes for the heavily-glycosylated spike protein, which mediates the binding of the virus to the Angiotensin-converting enzyme 2 (ACE2) resulting in the fusion of the viral and host cell membranes [15, 16]. The S glycoprotein is a trimeric class I fusion protein composed of three S subunits (S1, S2, and S3) where each has a receptor-binding domain (RBD) [17, 18]. The S glycoprotein is present in a metastable pre-fusion conformation, in which one of the three RBDs is rotated in a receptor-accessible conformation [19]. Upon binding of the S1 subunit to the host cell receptor, the pre-fusion trimer is destabilized, resulting in casting off the S1 subunit and conversion of the S2 subunit to a stable post-fusion conformation [20]. In order to engage the host cell receptor, substantial conformational movements of the RBD of the S1 subunit transiently hide or expose the determinants of receptor binding resulting into receptor-inaccessible (down) or -accessible (up) states, respectively [19, 21-23]. The S gene is divergent with <75% nucleotide sequence similarity when compared to all previously described SARSr-CoVs; thus it provides a specific marker for SARS-CoV-2 [7].

The M glycoprotein supports the viral envelope and is the most abundant structural component with a short N-terminus and a long C-terminus domains on the outside and inside of the virus, respectively [24]. M protein plays a crucial role in the intracellular formation of virus particles; however, in the absence of the S protein the virus is nonvirulent [24, 25]. The E protein is a small transmembrane comprised of three domains and functions as an ion-selective viroporin [26]. The binding motif of the E protein is involved in host cell processes and SARS-CoV pathogenesis, where it acts as a protein-protein interaction module [27]. The N protein forms helically symmetric nucleocapsid through binding to the RNA genome [28]. The phosphorylation by glycogen synthase kinase 3 (GSK3) in cells infected with SARS-CoV is important for viral replication since it activates the N protein [29]. The M, E and N proteins are more conserved among the CoVs than the S protein and are necessary for their general function [2]. Collectively, the four structural proteins are involved in encasing the RNA and/or in protein assembly, budding, envelope formation, and pathogenesis [30, 31].

SARS-CoV-2 poses a serious threat to human health due to its high contagiousness and capability to infect a wide variety of organisms, including avian and mammalian species that are consumed as livestock [32-34]. Given the fact that the number of the cases are on the rise and there are no vaccines or effective drugs available, the daily-life activities and working conditions of billions of people worldwide have been immensely disrupted due to different forms of public health intervention measures, such as closure of workplaces and lockdowns in many cities. Besides the heavy toll on human life, the outbreak has detrimental socio-economic effects due to the soaring unemployment rates and shattering of world economy resulting from business closures and major restrictions on travel [35]. Therefore, ubiquitous, reliable and rapid testing procedures are particularly indispensable in solving the complex dynamics involved in SARS-CoV-2 infection and immunity.

A contemporary concern of the COVID-19 pandemic is the need for readily accessible, accurate, efficient and cost-effective diagnostic testing for the detection of SARS-CoV-2 and its associated antibodies in infected individuals. Viral tests rely on biomolecular assays for the detection of SARS-CoV-2 viral RNA using polymerase chain reaction (PCR)-based techniques or nucleic acid hybridization-related strategies. PCR-/nucleic acid hybridization-based techniques including reverse transcriptase loop-mediated isothermal amplification (RT-LAMP) [38-40], CRISPR-based assays [41-43], Reverse-transcription quantitative PCR (RT-qPCR) remain in practice the most widely applied methods for the detection of RNA viruses.

There is still a need for cost effective methods of detecting viral RNA in a sample.

It is an object of the present invention to provide compositions and methods for detecting a nucleic acid from a pathogen in a sample.

It is another object of the present invention to provide compositions and methods for improved, rapid and sensitive detection of viral RNA in a sample.

It is a further object of the present invention to provide compositions and methods for one-step reactions that facilitate detection of a nuclei acid of interest in a sample.

### SUMMARY OF THE INVENTION

Compositions and methods for a one-step nucleic acid amplification and diagnostic methods based thereon are disclosed. The compositions and methods use isolated Thermus aquaticus DNA polymerase (Taq Pol) and Moloney Murine Leukemia Virus Reverse Transcriptase (MMLV-RT), in proportions and under reaction conditions that allow one-step amplification of a nucleic acid of interest.

In particular, disclosed are compositions containing a Moloney Murine Leukemia Virus Reverse Transcriptase (MMLV-RT) and a Thermus aquaticus DNA polymerase (Taq Pol) in effective amounts and in a buffer effective for one-step RT-PCR. In some embodiments, the compositions contain the MMLV-RT and Taq Pol enzymes in specific ratios, such as a 2:1 ratio of MMLV-RT to Taq Pol. The ratio can encompass the same or different concentration units for each of the enzymes. For example, in some embodiments, the ratio is defined by nanograms of MMLV-RT to units (U) of Taq Pol.

The amount of each enzyme contained in the compositions can vary. Typically, the compositions include from about 40 ng to about 80 ng MMLV-RT, from about 20 U to about 40 U Taq Pol, or combinations thereof.

In preferred embodiments, the compositions contain 60 ng MMLV-RT and 30 U Taq Pol.

A preferred MMLV-RT for use in the disclosed compositions and methods is an MMLV-RT having the amino acid sequence of SEQ ID NO:1. In some embodiments, a suitable MMLV-RT has an amino acid sequence containing at least 85% sequence identity to SEQ ID NO:1. Other MMLV-RT sequences are known however. See, for example, Genbank accession number: AAA66622.1, the contents of which are specifically incorporated by reference in their entirety.

An exemplary amino acid sequence of MMLV-RT is: AFPLERPDWDYTTQAGRNHLVHYRQLLLAGLQNAGRSPTNLAKVKGITQGP NESPSAFLERLKEAYRRYTPYDPEDPGQETNVSMSFIWQSAPDIGRKLGRL EDLKSKTLGDLVREAEKIFNKRETPEEREERIRRETEEKEERRRTVDEQKE KERDRRRHREMSKLLATVVIGQEQDRQEGERKRPQLDKDQCAYCKEKGHWA KDCPKKPRGPRGPRPQTSLLTLGDXGGQGQDPPPEPRITLKVGGQPVTFLV DTGAQHSVLTQNPGPLSDKSAWVQGATGGKRYRWTTDRKVHLATGKVTHSF LHVPDCPYPLLGRDLLTKLKAQIHFEGSGAQVVGPMGQPLQVLTLNIEDEY RLHETSKEPDVSLGFTWLSDFPQAWAESGGMGLAVRQAPLIIPLKATSTPV SIKQYPMSQEARLGIKPHIQRLLDQGILVPCQSPWNTPLLPVKKPGTNDYR PVQDLREVNKRVEDIHPTVPNPYNLLSGLPPSHQWYTVLDLKDAFFCLRLH PTSQPLFAFEWRDPEMGISGQLTWTRLPQGFKNSPTLFDEALHRDLADFR (SEQ ID NO:26, GenBank: AAA66622.1). Thus, in some embodiments, the MMLV-RT used in the compositions and methods has at least 85%, 90%, 95%, 96%, 97%, 98%, 99%, or 100% sequence identity with SEQ ID NO:26, or a nucleic acid sequence encoding SEQ ID NO:26.

A preferred Taq Pol for use in the disclosed compositions and methods is a Taq Pol having the sequence of SEQ ID NO:2 or an amino acid sequence containing at least 85% sequence identity to SEQ ID NO:2 (GenBank: AAA27507.1). In some embodiments, the MMLV-RT and/or Taq Pol include one or more tag sequences. The tag can be positioned at the N-terminus and/or C-terminus of each enzyme. Suitable tag sequences include, but are not limited to, His tags and Strep tags.

The compositions preferably include buffers that are suitable for storage of the enzymes and/or function of the enzymes (e.g., in one-step RT-PCR). Suitable components of the buffers include, without limitation, one or more salts, reducing agents, buffering agents, deoxynucleoside triphosphates (dNTPs), or combinations thereof. Exemplary salts that can; be included in the buffers are KCl, MgCl₂, and (NH₄)₂SO₄. In preferred embodiments, the dNTPs include dATP, dTTP, dGTP, and dCTP. In preferred embodiments, a suitable buffer includes about 50 mM Tris-HCl (pH 8.5), about 75 mM KCl, about 2 mM MgCl₂, about 1 mM DTT, about 200 μM dNTPs, and about 13.5 mM (NH₄)₂SO₄. Buffer components can be included in the following amounts: 20-50 mM Tris-HCl (pH 8.5); 75-150 mM KCl; 2-4 mM MgCl₂; 0.5-2 mM DTT; 200-500 μM dNTPs and 13.5 mM (NH₄)₂SO₄.

Also disclosed are methods of using the disclosed compositions. The compositions may be used to produce, analyze, quantitate, detect and otherwise manipulate nucleic acid molecules using RT-PCR procedures. In one embodiment, the method is a one-step RT-qPCR. An exemplary method of performing a one-step RT-PCR involves forming a mixture by combining an RNA sample/template and a plurality of primers with a disclosed composition and incubating the mixture under conditions sufficient to amplify one or more DNA molecules complementary to one or more portions of the RNA sample/template.

Also disclosed is method of detecting presence of a nucleic acid in a sample by combining the sample and plurality of primers specific to the nucleic acid with a disclosed composition under conditions sufficient for amplification of the nucleic acid, and detecting the nucleic acid amplification product, thereby detecting presence of the nucleic acid in the sample. In some embodiments, the nucleic acid is derived from a coronavirus, preferably a severe acute respiratory syndrome-related coronavirus, such as SARS-CoV-2.

Methods of diagnosis are also provided, such as methods of diagnosing infection with a pathogen and methods of detecting the presence of a pathogen of interest. In preferred embodiments, the pathogen is a virus (e.g., SARS-CoV-2). In particular, disclosed is a method of diagnosing a subject as infected with a virus by detecting the presence of a viral nucleic acid in a sample from the subject by performing any of the aforementioned methods. Typically, detecting an amplification product indicates that the subject is infected with the virus. The subject may or may not exhibit one or more symptoms of a disease, disorder, or condition associated with the virus. In some embodiments, the method further includes treating the subject, where the subject was diagnosed as infected with the virus. Preferably, the subject is human.

In any of the foregoing methods, the sample can be an RNA sample derived from mucus, sputum (processed or unprocessed), saliva, bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood. The sample can be one that is isolated from a subject that may have been exposed to or is suspected of having SARS-CoV-2. In some embodiments, the sample is processed to expose or isolate nucleic acids from sample before it is subjected to the detection, one-step RT-PCR, or other method.

Thus, in some embodiments, the methods include collecting one or more samples from a subject and/or extracting and purifying RNA from the samples. Typically, the purified RNAs are converted to DNA by a reverse transcription reaction using reverse transcriptase (Reverse transcription). At this stage, if the subject was infected with viral RNA for example, the complementary DNA (cDNA) fragments derived from the RNA viruses are generated. Then, the virus-originated DNA fragments are sufficiently amplified by the qPCR reaction to a detectable level (e.g., by a fluorescent signals). The one-step RT-qPCR platform can simultaneously achieve both the RT and qPCR reactions in a single tube.

Additional advantages of the disclosed methods and compositions will be set forth in part in the description which follows, and in part will be understood from the description, or can be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows a one-step RT-qPCR platform vs. two-step RT-qPCR in the context of work-flow to detect an RNA virus. After sampling the specimen from the patients, RNA materials need to be prepared by extraction and purification (Purification of RNAs). The purified RNAs are converted to DNAs by a reverse transcription reaction using reverse transcriptase (Reverse transcription). At this stage, if the patient was infected with RNA viruses, the complementary DNA (cDNA) fragments derived from the RNA viruses are generated. Then the virus-originated DNA fragments are sufficiently amplified by the qPCR reaction to a detectable level by the fluorescent signals (Amplification/Detection). While the one-step RT-qPCR platform can simultaneously achieve both the RT and qPCR reactions in a single tube, the two-step RT-qPCR needs two separate experimental setups, extra laboratory work, and have more chances for contamination by opening the tubes between the RT and PCR reactions.

FIGS. 2A-2D show purification of His-Taq Pol and C-His/Strep MMLV-RT. FIG. 2A shows a schematized summary of expression and purification procedures for His-Taq Pol and C-His/Strep MMLV-RT. FIG. 2B is a schematic representation of the constructs of the recombinant His-Taq Pol and C-His/Strep MMLV-RT expression vectors. CSPA promoter; cold-shock protein A promoter, RBS; ribosome binding site, 6X His; His-tag with 6 histidines, TEE; translational-enhancing element, Taq ORF; Taq Pol open reading frame, Polh; polyhedrin promoter, MMLV-RT ORF; MMLV-RT open reading frame, TEV; Tabacco Etch virus protease targeting site, 8X His; His-tag with 8 histidines, Strep; Strep-tag, polyA; SV40 late polyadenylation signal. FIG. 2C shows SDS-PAGE analysis of overexpressed His-Taq Pol in BL21(DE3) E. coli cells (left) and purified His-Pol. As a size control, native Taq Pol (N-Taq Pol) (Invitrogen, 18038-018) was applied by 1 μL (5 U). FIG. 2D shows SDS-PAGE analysis of purified C-His/Strep MMLV-RT expressed in the Sf9 cells. As a size control, SuperScript III (SSIII, Invitrogen, 18080051) was applied by 1 μL (200 U). FIG. 2E is a table showing the final yield of the purified protein from 1 L E. coli culture. FIG. 2F shows SDS-PAGE analysis of His-Taq and C-His/Strep MMLV-RT after the PCR reaction. After the PCR products were analyzed in FIG. 3C, the reaction mixture leftover (500 ng of C-His/Strep MMLV-RT vs. 20 units of His-Taq Pol) was subjected to the SDS-PAGE analysis (left panel). Fresh purified His-Taq Pol and C-His/Strep MMLV-RT were loaded as a size control (right panel). FIG. 2G shows standard titration curve of native Taq Pol's activity. The PCR assays were conducted with serially diluted N-Taq Pol (Thermofisher), and the standard titration curve of N-Taq Pol activity was plotted.

FIG. 3A shows activity assays of His-Taq Pol and N-Taq Pol by PCR. Purified His-Taq Pol was serially diluted with the storage buffer by the indicated dilution factors and subjected to the PCR reactions. N-Taq Pol is the same as in FIG. 2D and the standard titration curve made by N-Taq Pol is shown in FIG. 2G. FIG. 3B is a bar graph showing activity assays of C-His/Strep MMLV-RT and other commercially available reverse transcriptases. The first-strand cDNA was synthesized from SARS-CoV-2 N gene RNAs, and the 2019-nCoV_N3 qPCR assay was conducted. The yields of cDNA synthesis were shown in relative ones compared to that of SuperScript II. Error bars represent standard errors. FIG. 3C is a bar graph showing quantification of MMLV-RT inhibitory effect on Taq Pol activity in the PCR reaction. The indicated amounts of purified C-His/Strep MMLV-RT were added to the PCR premixture containing different units of Taq Pol. Error bars represent standard errors. FIG. 3D is a gel image showing the effect of ammonium sulfate on the inhibitory effect of MMLV-RT on the Taq Pol's activity in PCR. The different amounts of ammonium sulfate were added to the PCR reaction mixture without C-His/Strep MMLV-RT. Subsequently, 20 ng of C-His/Strep MMLV-RT was added to the reaction. FIG. 3E is a graph showing the effect of the DTT concentration on the baseline of the TaqMan based detection system. The one-step RT-qPCR reaction was performed with 1000 copies of the synthetic RNAs as a template. ΔRn; Rn is the fluorescence of the reporter dye divided by the fluorescence of a passive reference dye, ΔRn is Rn minus the baseline.

FIGS. 4A-4D are graphs showing determination of the effect of different proportions of Taq Pol and MMLV-RT in the one-step RT-qPCR reactions. The one-step RT-qPCR reactions were conducted using the synthetic RNAs with 10-fold serial dilutions (from 10 to 105 copies/μL) as a template with N1 or N2 primer sets. SuperScript III Platinum One-Step RT-qPCR kit was used as a control (FIG. 4A), 20 units (U) of His-Taq Pol and 40 ng/μL of C-His/Strep MMLV-RT (FIG. 4B), 30 U and 60 ng/μL (FIG. 4C), and 40 U and 80 ng/μL (FIG. 4D) were used. The mixtures were subjected to the reactions and the Ct values were plotted against the threshold cycle. Each plot represents the mean of 3 replicated Ct values with each RNA sample. The coefficient of determination (R²) and the equation of the regression curve (y) were calculated and shown in each panel.

FIGS. 5A-5C show elution profiles and SDS-PAGE gels of C-His/Strep MMLV-RT and His-Taq Pol from SEC. The elution profiles from size-exclusion chromatography (SEC) and SDS-PAGE gels are shown for the mixture of C-His/Strep MMLV-RT and His-Taq (FIG. 5A), His-Taq only (FIG. 5B), and C-His/Strep MMLV-RT (FIG. 5C). The protein samples were mixed with the PCR reaction buffer and incubated on ice for 10 minutes. And the incubated samples were loaded onto the Superdex 200 10/30 GL column (GE Healthcare).

### DETAILED DESCRIPTION OF THE INVENTION

The disclosed compositions and method may be understood more readily by reference to the following detailed description of particular embodiments and the Example included therein and to the Figures and their previous and following description.

Notwithstanding the facts that one-step RT-qPCR is well-suited for the diagnosis of COVID-19 and that there are many available commercial one-step RT-qPCR kits in the market, their high cost may impose financial challenges for mass screening. Cost effective methods that employ compositions containing Moloney Murine Leukemia Virus Reverse Transcriptase (MMLV-RT) and Thermus aquaticus DNA polymerase (Taq Pol) are disclosed.

### I. Definitions

“Unit” or “U” when used in context of an enzyme refers to an amount of enzyme required to convert a given amount of reactant to a product using a defined time and temperature.

As used herein, the term “detect”, “detecting”, “determine” or “determining” generally refers to obtaining information. Detecting or determining can utilize any of a variety of techniques available to those skilled in the art, including for example specific techniques explicitly referred to herein. Detecting or determining may involve manipulation of a physical sample, consideration and/or manipulation of data or information, for example utilizing a computer or other processing unit adapted to perform a relevant analysis, and/or receiving relevant information and/or materials from a source. Detecting or determining may also mean comparing an obtained value to a known value, such as a known test value, a known control value, or a threshold value. Detecting or determining may also mean forming a conclusion based on the difference between the obtained value and the known value.

As used herein, the term “sample” refers to body fluids, body smears, cells, tissues, organs or portion thereof isolated from a subject. A sample may be a single cell or a plurality of cells. A sample may be a specimen obtained by biopsy (e.g., surgical biopsy). A sample may be one or more of cells, tissue, serum, plasma, urine, spittle, sputum, stool, swab, blood, other bodily fluid, or exudate. In some embodiments, a sample includes nucleic acids, for example, viral DNA, viral RNA, or cDNA reverse transcribed from viral RNA. The sample can be used directly (e.g., fresh or frozen), or can be manipulated prior to use, for example, by heat-treatment, purification of nucleic acids, fixation (e.g., using formalin), and/or embedding in wax (such as FFPE tissue samples).

The terms “contact”, “contacting” or “combining” describe placement in physical association for example, in solid and/or liquid form. For example, contacting or combining can occur in vitro with one or more primers and/or probes and a biological sample (such as a sample including nucleic acids) in solution.

“Amplification” or “amplifying” refers to increasing the number of copies of a nucleic acid molecule, such as a gene, fragment of a gene, or other genomic region, for example at least a portion of an SARS-CoV-2 nucleic acid molecule. The products of an amplification reaction are called amplification products or amplicons. Amplification techniques include recombinase polymerase amplification (RPA), polymerase chain reaction (PCR), real-time PCR, quantitative real-time PCR (qPCR), reverse transcription PCR (RT-PCR), quantitative RT-PCR (qRT-PCR), loop-mediated isothermal amplification (LAMP), reverse-transcriptase LAMP (RT-LAMP), strand displacement amplification, transcription-free isothermal amplification, repair chain reaction amplification, ligase chain reaction amplification, gap filling ligase chain reaction amplification, and coupled ligase detection and PCR.

As used herein, the term “diagnosing” refers to identifying the nature of a disease or condition that a subject may be suffering from. As used herein, the term “diagnosis” refers to the determination and/or conclusion that a subject suffers from a particular disease or condition or is infected with a virus. The term “diagnosing” may denote the virus' or disease's identification (e.g., by an authorized physician).

As used herein, the term “primer” refers to an oligonucleotide, which is capable of acting as a point of initiation of nucleic acid synthesis when placed under conditions in which synthesis of a primer extension product which is complementary to a target nucleic acid strand is induced, e.g., in the presence of different nucleotide triphosphates and a polymerase in an appropriate buffer (“buffer” includes pH, ionic strength, cofactors etc.) and at a suitable temperature. The primer is preferably single-stranded. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the template. Primer includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of a double-stranded DNA (dsDNA) fragment. A “reverse primer” anneals to the sense-strand of a dsDNA fragment.

As used herein, the term “subject” refers to any individual, organism or entity. The subject can be a vertebrate, for example, a mammal. Thus, the subject can be a human. A subject may be a non-human primate, domestic animal, farm animal, or a laboratory animal. For example, the subject may be a dog, cat, goat, horse, pig, mouse, rabbit, or the like. The subject may be a human. The subject may be healthy or suffering from or susceptible to a disease, disorder or condition. A patient refers to a subject afflicted with a disease or disorder. The term “patient” includes human and veterinary subjects.

“Treatment” or “treating” means to administer a composition to a subject with an undesired condition (e.g., COVID-19). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. In some specific embodiments, treatment means to administer a composition or therapy in an amount sufficient to reduce, alleviate or ameliorate one or more symptoms of a disorder, disease, or condition being treated. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, “identity,” as known in the art, is a relationship between two or more polynucleotide or polypeptide sequences, as determined by comparing the sequences. In the art, “identity” also means the degree of sequence relatedness between the polynucleotide or polypeptide as determined by the match between strings of such sequences. “Identity” can also mean the degree of sequence relatedness of a polynucleotide or polypeptide compared to the full-length of a reference polynucleotide or polypeptide. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters can used to determine the identity for the polynucleotides or polypeptides of the present disclosure.

By way of example, a polynucleotide or polypeptide sequence may be identical to the reference sequence, that is be 100% identical, or it may include up to a certain integer number of nucleotides or amino acid alterations as compared to the reference sequence such that the % identity is less than 100%. Such alterations are selected from: at least one deletion, substitution, including conservative and non-conservative substitution, or insertion, and wherein said alterations may occur at the 5′ or 3′ end of the polynucleotide, or amino- or carboxy-terminal positions of the reference polypeptide sequence or anywhere between those terminal positions, interspersed either individually among the nucleic acids or amino acids in the reference sequence or in one or more contiguous groups within the reference sequence. The number of nucleotide or amino acid alterations for a given % identity is determined by multiplying the total number of nucleic acids or amino acids in the reference polynucleotide or polypeptide by the numerical percent of the respective percent identity (divided by 100) and then subtracting that product from said total number of nucleic acids or amino acids in the reference polynucleotide or polypeptide.

As used herein, the term “effective amount” means a quantity sufficient to provide a desired effect (e.g., catalysis of nucleic acid synthesis).

As used herein, the terms “polypeptide”” and “protein” are used interchangeably to refer to a polymer of amino acid residues. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “host cell” refers to a cell into which a recombinant vector can be introduced.

As used herein, “transformed” and “transfected” encompass the introduction of a nucleic acid (e.g. a vector) into a cell by a number of techniques known in the art.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other embodiments the values may range in value either above or below the stated value in a range of approximately +/−5%; in other embodiments the values may range in value either above or below the stated value in a range of approximately +/−2%; in other embodiments the values may range in value either above or below the stated value in a range of approximately +/−1%. The preceding ranges are intended to be made clear by context, and no further limitation is implied.

### II. Compositions

Typically, the compositions (e.g., suitable for one-step amplification of a nucleic acid of interest) include one or more enzymes. Suitable enzymes include RNA-dependent DNA polymerases (also referred to as reverse transcriptases; RT) and DNA-dependent DNA polymerases (also called DNA polymerases). Preferably, the compositions contain a reverse transcriptase and a DNA polymerase in effective amounts and in a buffer effective for one-step RT-PCR.

A. Reverse Transcriptase

The term “reverse transcriptase” describes a class of enzymes characterized as RNA-dependent DNA polymerases. All known reverse transcriptases require a primer to synthesize a DNA transcript from an RNA template. Historically, reverse transcriptase has been used primarily to transcribe mRNA into cDNA.

In preferred embodiments, the reverse transcriptase is a Moloney Murine Leukemia Virus Reverse Transcriptase (MMLV-RT). MMLV-RT is a 75 kDa, monomeric RNA-dependent DNA polymerase that lacks DNA endonuclease activity and has a low RNase H activity [46-49]. MMLV-RT is commonly used to synthesize cDNA from ssRNA, ssDNA, or RNA:DNA hybrid templates [49].

Protein and nucleic acid sequences of reverse transcriptases, including MMLV-RT, are known in the art. See, for example, Genbank accession number: AAA66622.1; NP_955591.1, the contents of which are specifically incorporated by reference in their entirety.

An exemplary amino acid sequence of MMLV-RT is: NP_955591.1 p80 RT [Moloney murine leukemia virus] LNIEDEHRLHETSKEPDVSLGSTWLSDFPQAWAETGGMGLAVRQAP LIIPLKATSTPVSIKQYPMSQEARLGIKPHIQRLLDQGILVPCQSPWNT PLLPVKKPGTNDYRPVQDLREVNKRVEDIHPTVPNPYNLLSGLPPSH QWYTVLDLKDAFFCLRLHPTSQPLFAFEWRDPEMGISGQLTWTRLP QGFKNSPTLFDEALHRDLADFRIQHPDLILLQYVDDLLLAATSELDCQ QGTRALLQTLGNLGYRASAKKAQICQKQVKYLGYLLKEGQRWLTE ARKETVMGQPTPKTPRQLREFLGTAGFCRLWIPGFAEMAAPLYPLTK TGTLFNWGPDQQKAYQEIKQALLTAPALGLPDLTKPFELFVDEKQG YAKGVLTQKLGPWRRPVAYLSKKLDPVAAGWPPCLRMVAAIAVLT KDAGKLTMGQPLVILAPHAVEALVKQPPDRWLSNARMTHYQALLL DTDRVQFGPVVALNPATLLPLPEEGLQHNCLDILAEAHGTRPDLTDQ PLPDADHTWYTDGSSLLQEGQRKAGAAVTTETEVIWAKALPAGTSA QRAELIALTQALKMAEGKKLNVYTDSRYAFATAHIHGEIYRRRGLLT SEGKEIKNKDEILALLKALFLPKRLSIIHCPGHQKGHSAEARGNRMAD QAARKAAITETPDTSTLLI (SEQ ID NO:1). Thus, in some embodiments, the MMLV-RT used in the compositions and methods has at least 85%, 90%, 95%, 96%, 97%, 98%, 99%, or 100% sequence identity with SEQ ID NO:1, or a nucleic acid sequence encoding SEQ ID NO:1.

In some embodiments the reverse transcriptase (such as MMLV-RT) is a thermostable enzyme. As used herein, the term “thermostable enzyme” refers to an enzyme which is stable to heat and catalyzes (facilitates) combination of the nucleotides in the proper manner to form the primer extension products that are complementary to each nucleic acid strand. A thermostable enzyme is not irreversibly denatured (or inactivated) when subjected to the elevated temperatures necessary for certain steps in the amplification procedure.

Since high reaction temperature reduces RNA secondary structures and non-specific primer binding, it is desirable in cDNA synthesis. As such, improvement of the thermostability of RT has been an important subject. Studies have shown that the thermostability of MMLV-RT can be improved by eliminating the RNase H activity (Kotewicz, et al., Gene, 35, 249-258 (1985); Gerard, et al., Nucleic Acids. Res., 30, 3118-3129 (2002); Mizuno, et al., Biosci. Biotechnol. Biochem., 74, 440-442 (2010)), by site-directed mutagenesis to generate a variant called MM3 (E286R/E302K/L435R) in which negatively charged or hydrophobic residues thought to interact with a template-primer are replaced with positively charged ones (Yasukawa, et al., J. Biotechnol., 150, 299-306 (2010)); by generating a V433R variant in which Valine 433 at the molecular surface is replaced with Arginine (Konishi, et al., Biosci. Biotechnol. Biochem., 78, 147-150 (2014)); or by generating the sextuple variant MM3.14 (A32V/L72R/E286R/E302K/W388R/L435R; Baba, et al., Protein Eng. Des. Sel., 30(8):551-557 (2017)).

Thus, native (wild-type) MMLV-RT or a variant, derivative, or active fragment thereof, including any of the above discussed or otherwise known thermostable MMLV RT variants can be used in accordance with the disclosed compositions and methods.

The reverse transcriptase (e.g., MMLV-RT) preferably has an optimum temperature at which it functions. An exemplary optimum temperature range includes from about 37° C. to about 45° C. (e.g., 42° C.). The disclosed purified MMLV-RT is robust enough and supports cDNA synthesis at comparable levels to that of commercially available reverse transcriptases.

B. DNA Polymerase

The compositions also include one or more DNA polymerases. Preferably, the DNA polymerase is a thermostable enzyme (e.g., not being irreversibly denatured or inactivated when subjected to the elevated temperatures necessary for certain steps in an amplification procedure). The DNA polymerase enzyme may be obtained from any source and may be a native or recombinant protein. For example, suitable DNA polymerases can be isolated from natural or recombinant sources, by techniques that are well-known in the art (see WO 92/06200, WO 96/10640, U.S. Pat. Nos. 5,455,170 and 5,466,591, the disclosures of all of which are incorporated herein by reference), from a variety of thermophilic bacteria that are available commercially (for example, from American Type Culture Collection, Rockville, Md.) or may be obtained by recombinant DNA techniques.

Suitable for use as sources of thermostable DNA polymerases or the genes thereof for expression in recombinant systems are the thermophilic bacteria Thermus thermophilus, Thermococcus litoralis, Pyrococcus furiosus, Pyrococcus woosii and other species of the Pyrococcus genus, Bacillus sterothermophilus, Sulfolobus acidocaldarius, Thermoplasma acidophilum, Thermus flavus, Thermus ruber, Thermus brockianus, Thermotoga neapolitana, Thermotoga maritima, Thermus aquaticus, Thermus lacteus, Thermus rubens, and other species of the Thermotoga genus, Methanobacterium thermoautotrophicum, Methanothermus fervidus, Bacillus stearothermophilus, and mutants, variants or derivatives thereof. It is to be understood, however, that thermostable DNA polymerases from other organisms may also be used.

A particularly preferred thermostable DNA polymerase for use in the compositions and methods is a Thermus aquaticus DNA polymerase (Taq Pol). Taq Pol is commercially available or can be isolated from its natural source, the thermophilic bacterium Thermus aquaticus, or be recombinantly generated. Methods for producing mutants and derivatives of thermophilic DNA polymerases are known in the art. Various strains of Thermus aquaticus are known and available for example, from the American Type Culture Collection (ATCC). Suitable strains of Thermus aquaticus for sourcing the DNA polymerase include YT-1 (ATCC 25104), Y-VII-51B (ATCC 25105), pFC85 [CMCC 3127] (ATCC 67421), and pWB254b [pWB254b/X7029] (ATCC 69244).

Taq Pol is a 94 kDa DNA-dependent-DNA polymerase that harbors 5′-3′ but not 3′-5′ exonuclease activity [50]. The unique 5′-3′ exonuclease activity of Taq Pol makes this enzyme especially suitable for TaqMan® real-time PCR assays in which the Taq Pol cleaves a dual-labeled probe annealed to the target sequence on cDNAs and releases the fluorescent reporter dye, thus resulting in increased fluorescence.

Protein and nucleic acid sequences of DNA polymerases, including Taq Pol, are known in the art. See, for example, Genbank accession number: AAA27507.1, the contents of which are specifically incorporated by reference in their entirety. An exemplary amino acid sequence of Taq Pol is:

(SEQ ID NO: 2, GenBank: AAA27507.1)
MRGMLPLFEPKGRVLLVDGHHLAYRTFHALKGLTTSRGEPVQAVYG
FAKSLLKALKEDGDAVIVVFDAKAPSFRHEAYGGYKAGRAPTPEDFP
RQLALIKELVDLLGLARLEVPGYEADDVLASLAKKAEKEGYEVRILT
ADKDLYQLLSDRIHVLHPEGYLITPAWLWEKYGLRPDQWADYRALT
GDESDNLPGVKGIGEKTARKLLEEWGSLEALLKNLDRLKPAIREKIL
AHMDDLKLSWDLAKVRTDLPLEVDFAKRREPDRERLRAFLERLEFG
SLLHEFGLLESPKALEEAPWPPPEGAFVGFVLSRKEPMWADLLALAA
ARGGRVHRAPEPYKALRDLKEARGLLAKDLSVLALREGLGLPPGDD
PMLLAYLLDPSNTTPEGVARRYGGEWTEEAGERAALSERLFANLWG
RLEGEERLLWLYREVERPLSAVLAHMEATGVRLDVAYLRALSLEVA
EEIARLEAEVFRLAGHPFNLNSRDQLERVLFDELGLPAIGKTEKTGKR
STSAAVLEALREAHPIVEKILQYRELTKLKSTYIDPLPDLIHPRTGRLH
TRFNQTATATGRLSSSDPNLQNIPVRTPLGQRIRRAFIAEEGWLLVAL
DYSQIELRVLAHLSGDENLIRVFQEGRDIHTETASWMFGVPREAVDP
LMRRAAKTINFGVLYGMSAHRLSQELAIPYEEAQAFIERYFQSFPKV
RAWIEKTLEEGRRRGYVETLFGRRRYVPDLEARVKSVREAAERMAF
NMPVQGTAADLMKLAMVKLFPRLEEMGARMLLQVHDELVLEAPKE
RAEAVARLAKEVMEGVYPLAVPLEVEVGIGEDWLSAKE.

Thus, in some embodiments, the Taq Pol used in the compositions and methods has at least 85%, 90%, 95%, 96%, 97%, 98%, 99%, or 100% sequence identity with SEQ ID NO:2, or a nucleic acid sequence encoding SEQ ID NO:2.

The DNA polymerase (e.g., Taq Pol) preferably has an optimum temperature at which it functions. For any disclosed thermostable polymerase, preferably, the enzyme will not become irreversibly denatured at about 90-100° C. Higher temperatures may also be tolerated as the buffer salt concentration and/or GC composition of the nucleic acid template is increased. An exemplary optimum temperature range for the polymerase activity of the DNA polymerase includes from about 40° C. to about 75° C. (e.g., 45-72° C.). The higher the temperature optimum for the enzyme's polymerase activity, the greater the specificity and/or selectivity of the primer-directed extension process. However, enzymes that are active below 40° C., e.g., at 37° C., are also contemplated for use provided that they are heat-stable. In some embodiments, the optimum temperature ranges from about 50° C. to 90° C., more preferably from about 60° C.-80° C. (e.g., 68° C.).

### Modifications and Tag Sequences

Various modifications to the enzymes can be made. In some embodiments, modifications to the primary structure of a disclosed enzyme by deletion, addition, or alteration of the amino acids incorporated into the sequence during translation can be made without destroying the activity of the enzymes. Besides N- or C-terminal deletions, individual residues in the amino acid peptide chain of a disclosed enzyme may be modified by oxidation, reduction, or other derivatization, and the enzyme may be cleaved to obtain fragments that retain activity. Such variants are contemplated for use in the disclosed compositions.

In some embodiments, the one or more enzymes (e.g., MMLV-RT and/or Taq Pol) contain one or more tag sequences and optionally, one or more linkers connecting the one or more tag sequences to the enzyme. Exemplary linker sequences include, but are not limited to GGGS (SEQ ID NO:24), GGGS (SEQ ID NO:25), (GGGS)₂ (SEQ ID NO:26); GGSA (SEQ ID NO:27). The tag can be positioned at the N-terminus and/or C-terminus of each enzyme. Suitable tag sequences include, but are not limited to, His tags and Strep tags. The “tag” refers to an amino acid sequence that is fused to the protein of interest to create the tagged protein. The tag sequence may be any peptide sequence encoded by a nucleic acid sequence. The tag sequence is fused in-frame to the protein coding sequence such that a fusion protein is generated. In-frame means that the open reading frame (ORF) of the sequence encoding the protein is maintained after the insertion of the tag sequence. In-frame insertions occur when the number of inserted nucleotides is divisible by three. In some embodiments, this may be achieved by adding a linker of any number of nucleotides to the tag protein encoding sequence as applicable.

The enzyme/protein may be tagged anywhere within the polypeptide sequence provided the function of the enzyme/protein is not compromised. Generally, the tag is positioned at the N- or C-terminus of the protein. The enzyme/protein may be tagged, for example, at the N-terminus of the protein. Alternatively, the enzyme/protein may be tagged at the C-terminus of the protein. In some embodiments, the enzyme/protein is tagged at both the N- and C-termini.

The enzyme/protein may be, for example, fused to the tag through a peptide linker. The sequence of the linker peptide can be chosen based on known structural and conformational contributions of peptide segments to allow for proper folding and preventing possible steric hindrance of the protein to be tagged and the tag polypeptide. Linker peptides are commonly used and known in the art, and may be from about 3 to about 40 amino acids in length.

The tag sequence may encode a variety of tags including, but not limited to, epitope tags, affinity tags, reporters, or combinations thereof. The tag may be, for example, an epitope tag. The epitope tag may contain a random amino acid sequence, or a known amino acid sequence. A known amino acid sequence may have, for example, antibodies generated against it, or there may be no known antibodies generated against the sequence. The epitope tag may be an antibody epitope tag for which commercial antibodies are available. Non-limiting examples of suitable antibody epitope tags are myc, AcV5, AU1, AU5, E, ECS, E2, FLAG, HA, Maltose binding protein (MBP), nus, Softag 1, Softag 3, Strep (e.g., Strep-II, Strep-III), SBP, Glu-Glu, HSV, KT3, S, S1, T7, V5, VSV-G, BCCP, calmodulin, and His. The polyhistidine or His-tag, usually contains 5-14 consecutive histidine residues (e.g., 6X His, 8X His). The sequences for these tags are known, and examples include, but are not limited to, HHHHHHHH (SEQ ID NO:15) (His tag), WSHPQFEK (strep tag) (SEQ ID NO:19); FLAG-tag (DYKDDDDK); 3X Flag (DYKDHDGDYKDHDIDYKDDDDK) (SEQ ID NO:16); HA-tag (YPYDVPDYA); (SEQ ID NO:24) Myc-tag (EQKLISEEDL) (SEQ ID NO:17); TC tag (CCPGCC) (SEQ ID NO:18) (Appl Microbiol. Biotechnol., 60:523-533 (2003) In one embodiment, the tag is a modified His-tag, modified to include additional amino acids such a strep-tag (WSHPQFEK) (SEQ ID NO:19) or the strep twin tag, which includes two strep tags separated by a linker and/or additional amino acids resulting from subcloning. A particularly preferred tag can be represented by the general formula SSAG-A-L₁-B-L₂-C, where A is a peptide cleave sequence, B is a protein tag as disclosed herein, L₁ and L₂ are optional first linkers with L₁ having the amino acid sequence SSS, for example, B and is a second protein tag. In one preferred embodiment, B and C can be a myc, AcV5, AU1, AU5, E, ECS, E2, FLAG, HA, Maltose binding protein (MBP), nus, Softag 1, Softag 3, Strep (e.g., Strep-II, Strep-III), SBP, Glu-Glu, HSV, KT3, S, S1, T7, V5, VSV-G, BCCP, calmodulin, and His and are preferably, His and strep tags. A particularly preferred tag sequence is SSAGENLYFQGSSSHHHHHHHHGGGSAWSHPOFEK (SEQ ID NO:20), where SSAG (SEQ ID NO:21): extra amino acid generated during the subcloning process; ENLYFQG (SEQ ID NO:22) is a TEV protease recognition site, HHHHHHHH (SEQ ID NO:15) is the His-tag; GGGSA (SEQ ID NO:23), is linker between His- and Strep-tags and WSHPQFEK (SEQ ID NO:19) is a strep tag. MMLV-RT made using the disclosed tag has comparable activity to the newer generation of engineered MMLV-RT (SuperScript II, Invitrogen). Additionally, the MMLV-RT has enough thermostable to work at 55° C. so that it can be used for other types of the applications, such as RT-LAMP.

Although the tag domain can be removed using TEV protease that cleaves its recognition site (ENLYFQG (SEQ ID NO:22)), the MMLV-RT can be used with the tag in place. Tobacco etch virus (TEV) protease is a 27-kDa catalytic domain of the polyprotein nuclear inclusion a (Nia) in TEV, which recognizes the specific amino acid sequence ENLYFQG/S and cleaves between Q and C/S.

Non-limiting examples of affinity tags include chitin binding protein (CBP), thioredoxin (TRX), poly(NANP), tandem affinity purification (TAP) tag, and glutathione-S-transferase (GST).

The enzyme can be tagged with more than one tag. For example, an enzyme may be tagged with at least one, two, three, four, five, six, seven, eight, or nine tags. For example, in some embodiments, the disclosed enzyme can be tagged at any desired terminus with a FLAG-HA tag or a His-Strep tag. More than one tag may be expressed as a single polypeptide fused to an enzyme/protein of interest. More than one tag fused to an enzyme may be expressed as a single polypeptide which can be cleaved into the individual tag polypeptides after translation. By way of a non-limiting example, 2A peptides of picornaviruses inserted between tag polypeptides or between tag polypeptide and the enzyme/protein of interest may result in the co-translational ‘cleavage’ of a tag and lead to expression of multiple proteins at equimolar levels.

Multiple tags are useful for the tandem affinity purification used to isolate recombinant proteins. Tandem affinity purification relies on two consecutive chromatographic steps that take advantage of the affinity tags placed at the end(s) of a target protein. This allows for efficient removal of contaminating proteins, including products of proteolytic degradation.

In some embodiments, the protein-tag fusion protein may contain one or more protease cleavage sites. The cleavage site(s) can be positioned between the protein and the tag and/or between multiple tags. The cleavage site can be used to remove affinity purification tags such as maltose-binding protein (MBP) or poly-histidine from fusion proteins. An exemplary cleavage site is the tobacco etch virus (TEV) protease recognition sequence, e.g., Glu-Asn-Leu-Tyr-Phe-Gln-(Gly/Ser). The TEV protease cleaves between the Gln and Gly/Ser residues.

C. Buffers

The disclosed enzyme compositions preferably include buffers and in some embodiments, can be stored in a buffer. Typically, the buffers provide appropriate pH and ionic conditions for the one or more enzymes. For example, a buffer can be an aqueous solution that provides optimal pH, ionic strength, cofactors, and the like for optimal enzyme activity. In some embodiments, the buffers are suitable for storage of the enzymes. In preferred embodiments, the buffers are suitable for one-step RT-PCR (e.g., the RT and PCR reactions can be performed in the same buffer). The buffers can also help relieve RT-mediated inhibition of DNA polymerase activity.

Suitable components of the buffers include, without limitation, one or more salts, reducing agents, buffering agents, deoxynucleoside triphosphates (dNTPs), chelating agents, detergents, preferably non-ionic detergents or combinations thereof. The one or more salts provide monovalent or divalent cations, such as, Mg2+, Mn2+, K+, NH4+, and Na+. Exemplary salts that can be included in the buffers are KCl, MgCl₂, NaCl, MnCl₂, NH₄Cl, MgSO4, (NH₄)₂SO₄, and magnesium acetate (e.g., Mg(C₂H₃O₂)₂; Mg(CH₃COO)₂·4H₂O). The concentration of the one or more salts can be in the range of from about 1 mM to about 500 mM, about 5 mM to about 250 mM, about 10 mM to about 200 mM, about 25 mM to about 150 mM, about 50 mM to about 100 mM, or about 60 mM. In some preferred embodiments, the buffer includes a combination of KCL and (NH₄)₂SO₄. The (NH₄)₂SO₄ can be included in the buffer in a concentration ranging from about 15-50 mM, preferably from about 10-35 mM, and/or KCL in a concentration range from about 50 to about 150 mM, preferably, between about 60 mM to about 90 mM, for example, 70, 75, 80 mM etc.

Other methods for storing the disclosed enzymes include storing a liquid preparation of each enzyme in a solution containing 50% (v/v) glycerol and a reducing agent such as dithiothreitol (DTT) or β-mercaptoethanol (βME) at −20° C. This method preserves the activity of the enzymes for many months with little loss of activity. Thus the stabilizing buffer can include 50% (v/v) glycerol and a reducing agent such as dithiothreitol (DTT) or β-mercaptoethanol (βME), or cryoprotetants such as sorbitol, xylitol, mannose, arabinose, sucrose, rhamnose, mannitol, trehalose, xylose, maltose, raffinose, and/or innulin. In some embodiments, the cryoprotectant is sorbitol. In some embodiments, the formulation comprises 20% sorbitol. In some embodiments, the concentration of cryoprotectant(s) is 1%-25%. Reverse transcriptase can also be successfully stabilized and lyophilized in a glycerol-free environment. TCEP (0.1-20 mM), NALC (n-acetyl-L-cysteine) (1-20 mM), and/or GSH/GSSG (1-20 mM). In some embodiments, the concentration of reducing agent(s) is 1-20 mM.

Inclusion of sugars like sucrose or trehalose, addition of polymer polyvinylpyrrolidone, and a reducing agent N-acetyl-L-cysteine protected the structure and activity of MMLV reverse transcriptase and resulted in the intact protein after a freeze-drying process [See U.S. Pat. No. 5,834,254].

Reducing agents, such as Dithiothreitol (DTT), β-mercaptoethanol, are typically used to stabilize enzymes and other proteins, which possess free sulfhydryl groups. DTT has been shown to restore activity lost by oxidation of these groups in vitro. DTT quantitatively reduces disulfide bonds and maintains monothiols in a reduced state. Suitable concentrations of DTT include from about 0.5 mM to about 1 mM.

Chelating agents include, but are not limited to divalent cation chelators such as EDTA (Ethylenediaminetetraacetic) and EGTA ((ethylene glycol-bis(β-aminoethyl ether)-N,N,N′,N′-tetraacetic acid).

Detergents include, but are not limited to Triton X-100, Nonidet P-40 octylphenoxypolyethoxyethanol), TWEEN®) 20 (Polysorbate 20). TWEEN®) 80 (Polysorbate 80), Brij™ 35 (Polyoxyethylene lauryl ether), Brij™ 68 (Polyoxyethylene (20) cetyl ether), TWEEN®) 85 (Polyoxyethylenesorbitan Trioleate), SYNPERONIC® (Poly(ethylene glycol)-block-poly(propylene glycol)-block-poly(ethylene glycol), Poly(propylene glycol)-block-poly(ethylene glycol)-block-poly(propylene glycol)) detergent, and/or Elugent™ (a mixture of alkyl glucosides) detergent. In some embodiments, the concentration of detergent(s) is 0.1 to 1%.

The compositions preferably contain one or more buffering agents. Suitable buffering agents are known in the art. The buffering agent can have a pH in the range of about 6 to 10 (e.g., a pH of 6.8 to 9, such as about pH 8.5). Suitable buffering agents include, without limitation, tris (e.g., Tris-HCl), tricine, bicine, HEPES, as well as other buffering agents known in the art. The concentration of the one or buffering agents can be in the range of from about 10 mM to about 100 mM. In some embodiments, the enzyme formulation comprises other stabilizers. In some embodiments, the other stabilizer(s) comprise arginine (in some modes, 0.1-1 M), MgCl₂ (in some modes, 1-10 mM), MgSO₄ (in some modes, 1-10 mM). TMAO (trimethylamine N-oxide) (in some modes, 0.1-1 M), PVP (polyvinylpyrrolidone) (in some modes, 0.1-2%), glycine (in some modes, 0.1-1 M), cysteine (in some modes, 0.1-1M), PVA (polyvinyl alcohol) (in some modes, 0.1-2%), PEG4000 (in some modes, 1-10%), PEG8000 (in some modes, 1-10%), Ficoll (in some modes, 1-10%).

An exemplary storage buffer is [20 mM Tris-HCl (pH 7.5), 100 mM NaCl, 0.01% NP-40, 0.1 mM EDTA, 50% (v/v) Glycerol]].

The compositions can further include one or more nucleotides (e.g., deoxynucleoside triphosphates (dNTPs)). The nucleotide components of the compositions serve as the “building blocks” for newly synthesized nucleic acids, being incorporated therein by the action of the reverse transcriptases or DNA polymerases. Examples of nucleotides suitable for use in the compositions include, but are not limited to, dUTP, dATP, dTTP, dCTP, dGTP, dITP, 7-deaza-dGTP, α-thio-dATP, α-thio-dTTP, α-thio-dGTP, α-thio-dCTP or derivatives thereof, all of which are available commercially from sources including Life Technologies, Inc. (Rockville, Md.), New England BioLabs (Beverly, Mass.) and Sigma Chemical Company (Saint Louis, Mo.). In preferred embodiments, the following dNTPs are included in the compositions: dATP, dTTP, dGTP, and dCTP. The nucleotides (e.g., dNTPs) may be unlabeled, or they may be detectably labeled by coupling them by methods known in the art, such as with radioisotopes (e.g., 3H, 14C, 32P or 35S), vitamins (e.g., biotin), fluorescent moieties (e.g., fluorescein, rhodamine, Texas Red, or phycoerythrin), chemiluminescent labels, dioxigenin and the like. Labeled dNTPs may also be obtained commercially, for example from Life Technologies, Inc. (Rockville, Md.) or Sigma Chemical Company (Saint Louis, Mo.).

The concentration of the dNTPs can vary. In some embodiments, the buffers or compositions contain a concentration of each dNTP of about 10-500 μM, about 10-300 μM, about 10-250 μM, or about 10-100 μM, and most preferably, a concentration of about 200 μM.

In preferred embodiments, a suitable buffer includes about 50 mM Tris-HCl (pH 8.5), about 75 mM KCl, about 2 mM MgCl₂, about 1 mM DTT, about 200 μM dNTPs, and about 13.5 mM (NH₄)₂SO₄.

The compositions can also contain one or more primers. In some embodiments, the primers facilitate the synthesis of a first DNA molecule complementary to all or a portion of an RNA template (e.g., a single-stranded cDNA molecule). Such primers may also be used to synthesize a DNA molecule complementary to all or a portion of a first DNA molecule, thereby forming a double-stranded cDNA molecule. Additionally, these primers may be used in amplifying nucleic acid molecules in accordance with the invention. Such primers include, but are not limited to, target-specific primers (e.g., gene-specific primers), oligo(dT) primers, random primers (e.g., random hexamers), or arbitrary primers. Additional primers that may be used according to the disclosed methods (e.g., DNA amplification) will be apparent to one of ordinary skill in the art.

Primers are typically at least 10, 15, 18, or 30 nucleotides in length. In some embodiments, primers are preferably between about 15 to about 30 nucleotides in length, and more preferably between about 20 to about 25 nucleotides in length. However, there is no standard primer length for optimal hybridization or amplification. An optimal length for a particular primer application may be readily determined by those of skill in the art.

D. Effective Amounts

The compositions contain the one or more enzymes and buffer in effective amounts for one-step RT-PCR. The effective amounts are sufficient to facilitate RT and PCR reactions. Suitable amounts of the various buffer components are discussed above.

In some embodiments, the concentration of DNA polymerases is determined as a ratio to the concentration of the enzyme having reverse transcriptase activity. Thus, the ratio of the reverse transcriptase enzymes to the DNA polymerase enzymes can be about 2:1 or less. Other suitable ratios of reverse transcriptase enzymes to DNA polymerases suitable for use in the compositions and methods will be apparent to one of ordinary skill in the art. The ratio can encompass the same or different concentration units for each of the enzymes. For example, in some embodiments, the ratio of RT to DNA polymerase is nanograms to units (U).

The amount of each enzyme contained in the compositions can vary. The working Example shows that at 100 ng or more, MMLV-RT completely inhibited activity of Taq Pol activity even at 20 units of Taq Pol. However, by reducing the amount of MMLV-RT to 40 ng, its inhibitory effect was overcome by using 20 units of Taq Pol. Thus, the compositions can include from about 40 ng to about 80 ng of a reverse transcriptase, from about 20 U to about 40 U of a DNA polymerase, or combinations thereof (e.g., 40 ng RT and 20 U DNA Pol; 80 ng RT and 40 U DNA Pol). Preferably, the compositions contain about 60 ng of a reverse transcriptase and about 30 U of a DNA polymerase.

### III. Methods of Making and Use

A. Methods of Making

Method of producing the polymerase and reverse transcriptase enzymes are also provided. In some embodiments, the methods include the steps of: (a) culturing a host cell containing a nucleic acid encoding the polymerase/RT in a suitable culture medium under suitable conditions to produce polymerase/RT; and (b) purifying the produced polymerase/RT.

Generally, the production of a recombinant enzyme such as RT or Taq polymerase involves the following: First, a DNA is obtained that encodes the enzyme or a fusion of the enzyme to an additional sequence that does not destroy its activity or to an additional sequence cleavable under controlled conditions (such as treatment with peptidase) to give an active protein. The coding sequence is then preferably placed in operable linkage with suitable control sequences (e.g., promoter) in a replicable expression vector. The vector is used to transform a suitable host and the transformed host is then cultured under favorable conditions to produce of the recombinant enzyme. Optionally, the enzyme is isolated from the medium or from the cells; recovery and purification of the protein may not be necessary in some instances, where some impurities may be tolerated.

Each of the foregoing steps can be done in a variety of ways. For example, the desired coding sequences may be obtained from genomic fragments and used directly in appropriate hosts. Construction of suitable vectors containing the desired coding and control sequences employs standard ligation and restriction techniques that are well understood in the art. Isolated plasmids, DNA sequences, or synthesized oligonucleotides are cleaved, tailored, and relegated in the form desired. The constructions for expression vectors operable in a variety of hosts are made using appropriate replicons and control sequences, as set forth below and known in the art. Suitable restriction sites can, if not normally available, be added to the ends of the coding sequence so as to provide an excisable gene to insert into these vectors. The control sequences, expression vectors, and transformation methods are dependent on the type of host cell used to express the gene. Generally, prokaryotic (e.g., bacteria), yeast, insect or mammalian cells are useful as hosts. A particularly preferred embodiment for production of the disclosed enzymes is exemplified below.

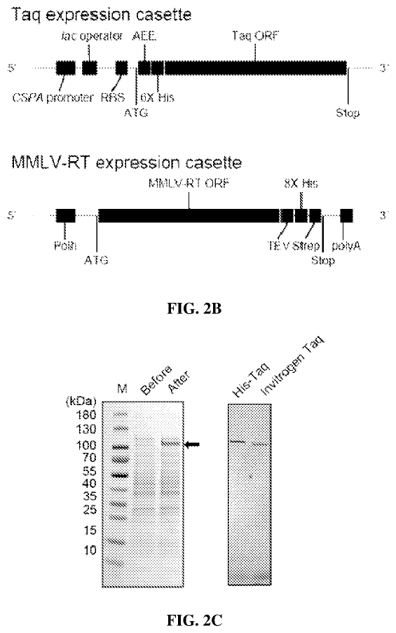

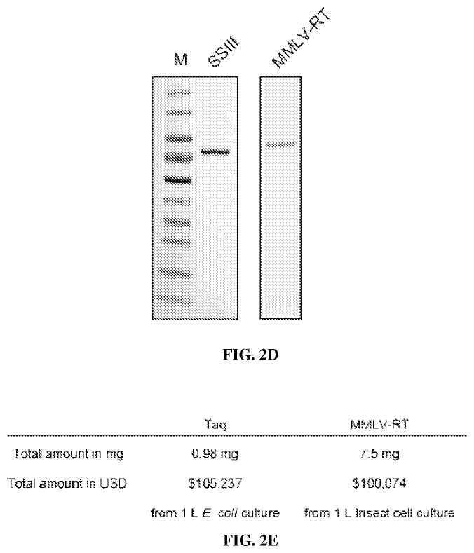

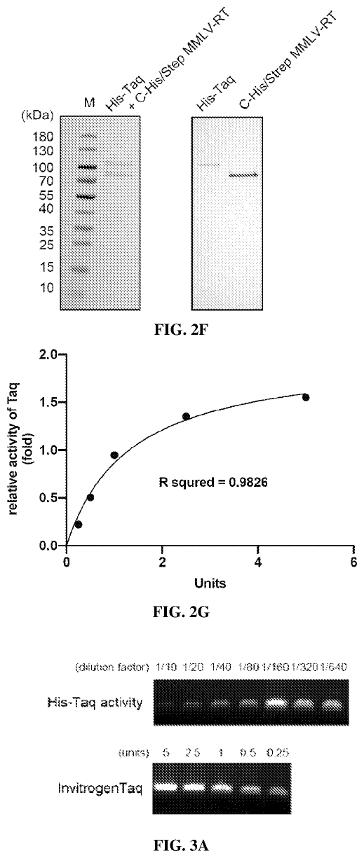

In some embodiments, the methods include using tagged proteins for both Taq Pol (His-Taq Pol) and MMLV-RT (C-His/Strep MMLV-RT) to enhance the expression in a host system such as E. coli and Sf9 cells, respectively, and to make use of the affinity chromatography in the protein purification procedures. The quantities of Taq Pol and MMLV-RT produced by the disclosed protein expression and purification schemes at the laboratory-scale are satisfactory and do not require production upscaling to an industrial-scale facility. The disclosed protein production platform provides a simpler follow-up example for groups in research institutes with limited-resources. Exemplary preferred expression and purification protocols are outlined in FIG. 2A and described in detail in the Materials and Methods section of the working Example.

The modified Polymerase/RT gene can be included in an expression cassette and/or cloned into a suitable expression vector by standard molecular cloning techniques. Such expression cassettes or vectors often contain sequences that assist initiation and termination of transcription (e.g., promoters and terminators), and may contain selectable markers. Cassettes can also be comprised of plus or minus strand mRNA, and their expression may or may not include an amplification step before translation of the mRNA. The expression cassette or vector can be introduced in a suitable expression host cell which will then express the corresponding enzyme (e.g., Polymerase, RT). Particularly suitable expression hosts are bacterial expression host genera including Escherichia (e.g., Escherichia coli), Pseudomonas (e.g., P. fluorescens or P. stutzerei), Proteus (e.g., Proteus mirabilis), Ralstonia (e.g., Ralstonia eutropha), Streptomyces, Staphylococcus (e.g., S. carnosus), Lactococcus (e.g., L. lactis), lactic acid bacteria or Bacillus (subtilis, megaterium, licheniformis, etc.).

The polymerase/RT protein may be expressed from the DNA, using expression vectors maintained within host cells. DNA cloning, manipulation and protein expression are all standard techniques in the art, and details of suitable techniques may be found in Sambrook et al., “Molecular cloning: A Laboratory Manual”.

Delivery vehicles can be used to introduce the genes encoding the Polymerase/RT into a host cell. The delivery vehicle can be a viral vector, for example a commercially available preparation, such as an adenovirus vector. The viral vector delivery can be via a viral system, such as a retroviral vector system which can package a recombinant retroviral genome. The recombinant retrovirus can then be used to infect and thereby deliver to the infected cells nucleic acid encoding a protein of interest. The exact method of introducing the altered nucleic acid into the host cell is, of course, not limited to the use of retroviral vectors. Other techniques are widely available for this procedure including the use of adenoviral vectors, adeno-associated viral (AAV) vectors, lentiviral vectors, pseudotyped retroviral vectors, and others described in (Soofiyani, et al., Advanced Pharmaceutical Bulletin, 3(2):249-255 (2013). Viruses can be modified to enhance safety, increase specific uptake, and improve efficiency (see, for example, Zhang, et al., Chinese J Cancer Res., 30(3):182-8 (2011), Miller, et al., FASEB J, 9(2):190-9 (1995), Verma, et al., Annu Rev Biochem., 74:711-38 (2005)).

Physical transduction techniques can also be used, such as liposome delivery and receptor-mediated and other endocytosis mechanisms (see, for example, Schwartzenberger et al., Blood, 87:472-478 (1996)). Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, nucleic acid or vectors encoding proteins of interest can be delivered in vivo by electroporation as well as by means of a sonoporation. During electroporation electric pulses are applied across the cell membrane to create a transmembrane potential difference, allowing transient membrane permeation and transfection of nucleic acids through the destabilized membrane (Soofiyani, et al., Advanced Pharmaceutical Bulletin, 3(2):249-255 (2013)). Sonoporation combines the local application of ultrasound waves and the intravascular or intratissue administration of gas microbubbles to transiently increase the permeability of vessels and tissues (Escoffre, et al., Curr Gene Ther., 13(1):2-14 (2013)). Expression or overexpression of the disclosed proteins can be accomplished with any of these or other commonly used gene transfer methods, including, but not limited to use of a gene gun.

In some embodiments, the genetic sequence can be stably inserted into the genome of the organism expressing the protein.

The protein of interest can be secreted into the extracellular or periplasmic space or expressed intracellularly. In some embodiments, the DNA-Polymerase/RT is expressed in a microbial host and the protein is secreted into the periplasmic or extracellular space. Cultures of the expressing organism are prepared at an appropriate volume with standard methods of fermentation. In a preferred embodiment, cultures for protein expression are inoculated from a cryo stock and the volume of the culture increased successively in the appropriate containers. In a preferred embodiment the cells are grown in a fermenter and optionally growth conditions such as pH, temperature, oxygen and/or nutrient supply are controlled. A first step of purification comprises the separation of cells from supernatant using one or more of several techniques, such as sedimentation, microfiltration, centrifugation, flocculation or other. In a preferred embodiment the method applied is microfiltration. In case of intracellular expression the cells are subjected to treatments that result in a release of the protein from the intracellular space. These treatments may comprise for example pressure, enzymatic treatment, osmotic shock, freezing, ultrasonic or other treatment to produce a cellular extract which may or may not be subjected to further purification.

The preferred purification method yields a purity of the protein of >30%. In a more preferred embodiment, the purity is of the protein is >50%, >60%, >70%, >80%, or >90%.

B. Methods of Use

Methods of using the compositions are provided. For example, the enzyme containing compositions can be used in methods of detecting presence of a nucleic acid in a sample and/or methods of diagnosis. The disclosed compositions are suitable for use in various nucleic acid amplification reactions (e.g., reverse transcriptase-polymerase chain reaction (RT-PCR)) and analysis of nucleic acids. In particular, disclosed are compositions containing a variety of components in various combinations. Such components include one or more enzymes having reverse transcriptase activity, one or more DNA polymerases, one or more primers, one or more nucleotides and a suitable buffer. These compositions may be used in the disclosed methods to produce, analyze, quantitate and otherwise manipulate nucleic acid molecules (e.g., using amplification reactions, such as a one-step RT-PCR procedure).

Various amplifying reactions are well known to one of ordinary skill in the art and include, but are not limited to PCR, RT-PCR, LCR, in vitro transcription, rolling circle PCR, OLA and the like. In some embodiments, multiple primers can be used in a multiplex PCR for detecting a set of more than one specific target molecules.

Preferably, the disclosed enzyme containing compositions can be used in a one-step assay, for various types of nucleic acid amplification reactions, for example, RT-PCR and RT-LAMP assays. Accordingly, the disclosed enzymes and compositions can be used in a method for the synthesis or amplification of a target nucleic acid sequence, which involves forming a reaction mixture including: (i) the target nucleic acid sequence; (ii) a composition having nucleic acid polymerase activity as described herein; and (iii) nucleoside-5′-triphosphates to support the nucleic acid polymerase activity; and (b) subjecting the reaction mixture to conditions to synthesise or amplify the target nucleic acid sequence.

The disclosed methods use a selection of defined conditions to circumvent the previously reported inhibition of Taq Pol by reverse transcriptase [51], allowing for a one-step RT-qPCR. A particularly preferred embodiment is demonstrated in the working Example below using SAR-CoV-2. In some embodiments, the disclosed Taq Pol (e.g., non-hot-start Taq Pol) performs amplification in the one-step RT-qPCR platform without drifting the baseline in the fluorescent detection scheme.

### i. One-Step RT-PCR

One-step RT-PCR involves including the RT step into the same tube as the PCR reaction, unlike the two-step RT-PCR method which involves creating cDNA first by means of a separate reverse transcription reaction and then adding the cDNA to the PCR reaction. Therefore, in the one-step RT-PCR, synthesis and PCR are carried out in the same reaction vessel in a common buffer. Some advantages to one-step real-time RT-PCR is that it is quicker to set up, less expensive to use, and involves less handling of samples, thereby reducing pipetting errors, contamination, and other sources of error. With the one-step method, gene-specific primers are used and both the RT and PCR occur in one reaction tube. The RNA from the original sample must be initially aliquoted for archival storage and future testing. Because specific primers typically anneal at higher temperatures than random primers, one-step protocols often use higher RT reaction temperatures than two-step workflows and may employ RTs that can tolerate higher reaction temperatures.

In RT-PCR, first, the reaction mixtures are incubated at a temperature sufficient to synthesize a DNA molecule complementary to all or portion of the RNA template. Such conditions typically range from about 20° C. to 75° C., more preferably from about 35° C. to 60° C. and most preferably from about 45° C. to about 55° C. After the reverse transcription reaction, the mixture is incubated at a temperature sufficient to amplify the synthesized DNA molecule. In general, the amplification process involves a chain reaction for producing, in exponential quantities relative to the number of reaction steps involved, at least one specific nucleic acid sequence given (a) that the ends of the required sequence are known in sufficient detail that primers can be synthesized which will hybridize to them, and (b) that a small amount of the sequence is available to initiate the chain reaction. The product of the chain reaction will be a discrete nucleic acid duplex with termini corresponding to the ends of the specific primers employed.

Preferably the amplification is accomplished via one or more polymerase chain reactions (PCRs). Preferred conditions for amplification include thermocycling, which may involve alternating heating and cooling of the mixture sufficient to amplify the DNA molecule. This can include alternating from a first temperature range of from about 90° C. to about 100° C., to a second temperature range of from about 45° C. to about 75° C., more preferably from about 50° C. to about 75° C. or from about 55° C. to about 75° C., and most preferably from about 65° C. to about 75° C. (e.g., about 68° C.). Each step of the thermocycling procedure can be performed for any length of time such as from about 15 seconds to about 2 minutes. The thermocycling may be performed any number of times, for example from about 5 to about 80 times, preferably, greater than about 10 times and most preferably, greater than about 20 times.

In some embodiments, the method of performing a one-step RT-PCR involves forming a mixture by combining an RNA sample/template and a plurality of primers with a disclosed composition and incubating the mixture under conditions sufficient to amplify one or more DNA molecules complementary to one or more portions of the RNA sample/template.

### ii. RT-LAMP

In some embodiments, the disclosed polymerase and RT and compositions thereof are used in reverse transcription coupled loop-mediated isothermal amplification (RT-LAMP) methods, to detect the presence of a pathogen of interest, preferably viral infections, for example, the presence of SARS-Co-V-2 RNA in a sample.

Loop-mediated isothermal amplification (LAMP) is a single-tube technique for the amplification of DNA, which may be combined with a reverse-transcription step to allow the detection of RNA (RT-LAMP). RT-LAMP requires primers, a reverse transcriptase enzyme, and a DNA polymerase enzyme having strand displacement activity for the amplification of RNA. Similar to RT-PCR, conventional RT-LAMP requires a reverse transcriptase enzyme to synthesize complementary DNA (cDNA) from RNA sequences. This cDNA can then be amplified using DNA polymerase.

RT-LAMP can be desirable because of the relatively low reaction temperature and no need for thermocycling equipment necessary for other methods like PCR. In conventional LAMP, four specially designed primers can recognize distinct target sequences on a template strand. Such primers bind only to these sequences which allows for high specificity. Out of the four primers involved, two of them are “inner primers” (FIP and BIP), designed to synthesize new DNA strands. The outer primers (F3 and B3) anneal to the template strand and also generate new DNA. These primers are accompanied by a DNA polymerase which can aid strand displacement and can release the newly formed DNA strands.

The BIP primer (in conventional methods, accompanied by a reverse transcriptase enzyme), can initiate the process by binding to a target sequence on the 3′ end of an RNA template and synthesizing a copy DNA strand. The B3 primer can also bind the 3′ end and along with a polypeptide having DNA polymerase activity (e.g., a mutant polymerase described herein) can simultaneously create a new cDNA strand while displacing the previously made copy. The double stranded DNA containing the template strand is no longer needed.

At this point, the single stranded copy can loop at the 3′ end as it binds to itself. The FIP primer can bind to the 5′ end of this single strand and accompanied by a polypeptide having DNA polymerase activity (e.g. the BR3 polymerase and mutants described herein), can synthesize a complementary strand. The F3 primer, with DNA polymerase, can bind to this end and can generate a new double stranded DNA molecule while displacing the previously made single strand.

This newly displaced single strand can act as the starting point for a LAMP cycling amplification. The DNA can have a dumbbell-like structure as the ends fold in and self-anneal. This structure can become a stem-loop when the FIP or BIP primer once again initiates DNA synthesis at one of the target sequence locations. This cycle can be started from either the forward or backward side of the strand using an appropriate primer. Once this cycle has begun, the strand can undergo self-primed DNA synthesis during the elongation stage of the amplification process. This amplification can take place in about an hour, under isothermal conditions between about 60-65° C.

### iii. Detection of Amplification Products

In any of the foregoing methods (e.g., one-step RT-PCR, RT-LAMP) following the amplification reaction, the amplification product(s) can be detected by any suitable method. For example, disclosed is method of detecting presence of a nucleic acid in a sample by combining the sample and plurality of primers specific to the nucleic acid with a disclosed composition under conditions sufficient for amplification of the nucleic acid, and detecting the nucleic acid amplification product, thereby detecting presence of the nucleic acid in the sample. The detection method may be quantitative, semi-quantitative, or qualitative. The particular nucleic acid (e.g., viral nucleic acid) may be determined in some cases by the band pattern observed on gel electrophoresis. Amplification products can also be detected using a colorimetric assay, such as with an intercalating dye (for example, propidium iodide, SYBR green, GelRed™, or GelGreen™ dyes). Amplification products can also be detected with a metal ion sensitive fluorescent molecule (for example, calcein, which is a fluorescence dye that is quenched by manganese ions and has increased fluorescence when bound to magnesium ions).

In some embodiments, a sample is identified as containing a viral nucleic acid (for example, the sample is “positive” for the virus) if one or more amplifications products are detected (e.g., by any suitable quantitative, semi-quantitative, or qualitative approach). In some embodiments, a sample is identified as containing a viral nucleic acid (for example is “positive” for the virus) if an increase in fluorescence is detected compared to a control (such as a no template control sample or a known negative sample).

In some embodiments fluorescence is used for the quantitative detection of one or more amplification products. Generally, this relies on use of a detection probe containing a fluorophore moiety and a quencher moiety, positioned in such a way that the hybridized state of the probe can be distinguished from the unhybridized state of the probe by an increase in the fluorescent signal from the nucleotide. The fluorophore and quencher molecules are incorporated into the probe in sufficient proximity such that the quencher quenches the signal of the fluorophore molecule when the probe is hybridized to its recognition sequence.

Cleavage of the probe by a DNA polymerase with 5′ nuclease activity results in separation of the quencher and fluorophore molecule, and the presence in increasing amounts of signal as nucleic acid sequences. A particular embodiment is the TaqMan® assay described below. At the start of real-time PCR, the temperature is raised to denature cDNA. During this step, the signal from the fluorescent dye on the 5′ end of the TaqMan probe is quenched by the NFQ on the 3′ end. In the next step, the reaction temperature is lowered to allow the primers and probe to anneal to their specific target sequences. The Taq DNA polymerase then synthesizes new DNA strands using unlabeled primers and the template. When the polymerase reaches a TaqMan probe, its endogenous 5′ nuclease activity cleaves the probe, separating the dye from the quencher. With each cycle of PCR, more dye molecules are released, resulting in an increase in fluorescence intensity proportional to the amount of amplicon synthesized.

### iv. Methods of Diagnosis and/or Treatment

Methods of diagnosis are also provided, such as methods of detecting or diagnosing infection with a pathogen or methods of detecting the presence of a pathogen of interest (e.g., a virus such as SARS-CoV-2).

The methods can provide rapid, fast and accurate results on the presence of a viral nucleic acid (e.g., SARS-CoV-2) in a sample. Typically, the method of diagnosing involves any of the methods for detecting a viral nucleic acid discussed above. For example, a method of diagnosing a subject for infection with a virus can include detecting the presence of a viral nucleic acid in a sample from the subject by performance of a one-step RT-PCR. In some embodiments, a method of diagnosing a subject as infected with a virus includes detecting the presence of a viral nucleic acid in a sample from the subject by performing any of the aforementioned methods of detection. Typically, detecting an amplification product or a plurality of amplification products indicates the subjects is infected with the virus. The detection can be qualitative or quantitative.

In a particular embodiment, the current or present exposure or infection with SARS-CoV-2 can be detected and/or diagnosed and/or treated using the disclosed compositions and methods. Typically, the presence and/or elevated amount of SARS-CoV-2 nucleic acid in a subject's biological sample as compared to a control (as determined by any disclosed method of detection for example) is indicative of current or past exposure or an active infection with SARS-CoV-2.

The subject may or may not exhibit symptoms of a disease, disorder, or condition associated with the virus. For example, a symptomatic subject may be suspected of having a particular viral infection. In such cases, the method of diagnosis can be used to confirm the etiology of the infection by detecting the presence of a particular viral nucleic acid in a sample from the subject. In some embodiments, the subject is asymptomatic, but can be suspected of having contact with a virus. In such cases, the method of diagnosis can be used to confirm exposure and/or infection with the virus.

The methods can be used to diagnose a viral infection at early stages. The early stages include asymptomatic or presymptomatic stages of infection, as well as days 0, 1, and 2 post symptom onset. In some embodiments, the methods have accuracy of greater than 90% specificity and greater than 90% sensitivity for detecting a viral infection (e.g., SARS-CoV-2) in the subject prior to onset of symptoms of infection, on the day of onset of symptoms of infection, or one day, two days, three days, four days, five days, six days, or seven days after onset of symptoms of infection.

Exemplary viruses and symptoms of illness stemming from infection by the viruses that are treatable by the disclosed methods are also provided. The virus is typically a coronavirus. Coronaviruses cause diseases in mammals and birds. In humans, coronaviruses can cause respiratory tract infections that can range from mild to lethal. Mild illnesses include some cases of the common cold, while more lethal varieties can cause SARS, MERS, and COVID-19 (i.e., caused by SARS-CoV-2).

Thus, in some embodiments, the subject may have one or more symptoms characteristic of SARS, MERS, or COVID-19. SARS (i.e., SAR-CoV) usually begins with flu-like signs and symptoms such as fever, chills, muscle aches, headache and occasionally diarrhea. After about a week, signs and symptoms include fever of 100.5 F (38 C) or higher, dry cough, shortness of breath, headache, muscular stiffness, loss of appetite, malaise, confusion, rash, or diarrhea, or any combination thereof.

Reported illnesses from COVID-19 (i.e., caused by SARS-CoV-2) have ranged from mild symptoms to severe illness and death for confirmed cases. The most common symptoms are fever, tiredness, dry cough, loss of taste or smell, sore throat, runny nose, congestion, vomiting, diarrhea and shortness of breath. These symptoms may appear 2-14 days after exposure. Symptoms differ with severity of disease. For example, fever, cough, and shortness of breath are more commonly reported among people who are hospitalized with COVID-19 than among those with milder disease (non-hospitalized patients). Atypical presentations occur often, and older adults and persons with medical comorbidities may have delayed presentation of fever and respiratory symptoms. In one study of 1,099 hospitalized patients, fever was present in only 44% at hospital admission but eventually developed in 89% during hospitalization. Fatigue, headache, and muscle aches (myalgia) are among the most commonly reported symptoms in people who are not hospitalized, and sore throat and nasal congestion or runny nose (rhinorrhea) also may be prominent symptoms. Many people with COVID-19 experience gastrointestinal symptoms such as nausea, vomiting or diarrhea, sometimes prior to developing fever and lower respiratory tract signs and symptoms. Loss of smell (anosmia) or taste (ageusia) preceding the onset of respiratory symptoms has been commonly reported in COVID-19 especially among women and young or middle-aged patients who do not require hospitalization. While many of the symptoms of COVID-19 are common to other respiratory or viral illnesses, anosmia appears to be more specific to COVID-19. In critical cases, respiratory failure, shock, or multiorgan system dysfunction is common.

Most people confirmed to have MERS-CoV infection have had severe respiratory illness with symptoms of fever, cough, and/or shortness of breath. Some people also exhibit diarrhea and nausea/vomiting. For many people with MERS, more severe complications followed, such as pneumonia and kidney failure. Some infected people had mild symptoms (such as cold-like symptoms) or no symptoms at all.

In some embodiments, the subject has an underlying condition such as asthma, heart disease, diabetes, cancer, chronic lung disease, chronic heart disease, chronic kidney disease, an autoimmune disease, or a combination thereof. Preferably, the subject is human.

Any of the disclosed methods can be combined with a method of treatment. In some embodiments, upon diagnosis as infected with a virus, the subject is further treated. In preferred embodiments, the method of treatment includes administering the subject an effective amount of an anti-viral therapy (e.g., remdesivir), analgesic therapy (e.g., ibuprofen, acetaminophen), corticosteroid therapy (e.g., dexamethasone, prednisone, methylprednisolone, or hydrocortisone), fever reducers, cough suppressants, and/or respiratory assistance (e.g., supplemental oxygen or mechanical ventilation).

### Samples

The methods may be used for any purpose for which detection of nucleic acids is desirable, including diagnostic and prognostic applications, such as in laboratory and clinical settings. Appropriate samples include any conventional biological samples, including clinical samples obtained from a human or veterinary subject. Suitable samples include all biological samples useful for detection of infection in subjects, including, but not limited to, cells (such as buccal cells or peripheral blood mononuclear cells), tissues, autopsy samples, bone marrow aspirates, bodily fluids (for example, blood, serum, plasma, urine, cerebrospinal fluid, middle ear fluids, bronchoalveolar lavage, tracheal aspirates, sputum, nasopharyngeal aspirates, oropharyngeal aspirates, or saliva), oral swabs, eye swabs, cervical swabs, vaginal swabs, rectal swabs, stool, and stool suspensions.

In preferred embodiments, the sample is mucus, sputum (processed or unprocessed), bronchial alveolar lavage (BAL), bronchial wash (BW), cerebrospinal fluid (CSF), urine, tissue (e.g., biopsy material), rectal swab, nasopharyngeal aspirate, nasopharyngeal swab, throat swab, saliva, feces, mucosal excretions, plasma, serum, or whole blood. In some embodiments, the sample is a nucleic acid isolated and/or derived from any of the foregoing biological samples.

Generally, the sample is obtained non-invasively, such as by swabbing, scraping, collecting, drawing, or draining.

The sample can be used directly or can be processed, such as by adding solvents, preservatives, buffers, or other compounds or substances. In some examples, nucleic acids are isolated and/or derived from the sample. In other examples, isolation of nucleic acids from the sample is not necessary prior to use and the sample (such as a plasma or serum sample) is used directly (without nucleic acid extraction, but potentially with heat-treatment or other processing step). In some embodiments, the sample can be pre-treated with a lysis buffer, but nucleic acids are not isolated prior to use.

Samples also include isolated nucleic acids, such as DNA or RNA isolated from a biological specimen from a subject, a viral isolate, or other source of nucleic acids. The sample can also include DNA that is reverse transcribed from RNA isolated or extracted from a biological specimen from a subject, a viral isolate, or other source of nucleic acids. Any nucleic acid sequence, in purified or nonpurified form, can be utilized as the sample provided it contains or is suspected to contain the specific nucleic acid sequence desired. The DNA or RNA may be single-stranded or double-stranded. In some embodiments, DNA-RNA hybrids may be used.

Methods for extracting nucleic acids such as RNA or DNA from a sample are known to one of skill in the art. Such methods will depend upon, for example, the type of sample in which the nucleic acid is found. Nucleic acids can be extracted using standard methods. For instance, rapid nucleic acid preparation can be performed using commercially available reagents/kit, such as kits and/or instruments from Invitrogen (TRIzol) Zymo Research (Direct-Zol RNA Miniprep kit), Qiagen (such as QiaAmpO, DNEasy® or RNEasy® kits), Roche Applied Science (such as MagNA Pure kits and instruments), Thermo Scientific (KingFisher mL), bioMerieux (Nuclisens® NASBA Diagnostics), or Epicentre (Masterpure™ kits). In some embodiments, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987).

The specific nucleic acid sequence to be amplified may be only a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, or a portion of a nucleic acid sequence due to a particular microorganism which organism might constitute only a very minor fraction of a particular biological sample. The starting nucleic acid sequence may contain more than one desired specific nucleic acid sequence which may be the same or different. Therefore, the amplification process is useful not only for producing large amounts of one specific nucleic acid sequence, but also for amplifying simultaneously more than one different specific nucleic acid sequence located on the same or different nucleic acid molecules.

### Exemplary Viruses

The disclosed methods are suitable for the detection of any virus or nucleic acid therefrom. In some embodiments, the virus is a coronavirus, such as a severe acute respiratory syndrome-related coronavirus (e.g., SARS-CoV or SARS-CoV-2). The current classification of coronaviruses recognizes 39 species in 27 subgenera, five genera and two subfamilies that belong to the family Coronaviridae, suborder Cornidovirineae, order Nidovirales and realm Riboviria (Coronaviridae Study Group of the International Committee on Taxonomy of Viruses, Nat Microbiol 2020. DOI: 10.1038/s41564-020-0695-z). They are enveloped viruses with a positive-sense single-stranded RNA genome and a nucleocapsid of helical symmetry. The genome size of coronaviruses ranges from approximately 26 to 32 kilobases, one of the largest among RNA viruses.

Coronavirus species and representative viruses thereof include [representative virus (of species)]: SARSr-CoV BtKY72 (Severe acute respiratory syndrome-related coronavirus), SARS-CoV-2 (Severe acute respiratory syndrome-related coronavirus), SARSr-CoV RaTG13 (Severe acute respiratory syndrome-related coronavirus), SARS-CoV PC4-227 (Severe acute respiratory syndrome-related coronavirus), SARS-CoV (Severe acute respiratory syndrome-related coronavirus), Bat-Hp-BetaCovC (Bat Hp-betacoronavirus Zhejiang2013), Ro-BatCoV GCCDC1 (Rousettus bat coronavirus GCCDC1), Ro-BatCoV HKU9 (Rousettus bat coronavirus HKU9), Ei-BatCoV C704 (Eidolon bat coronavirus C704), Pi-BatCoV HKU5 (Pipistrellus bat coronavirus HKU5), Ty-BatCoV HKU4 (Tylonycteris bar coronovirus HKU4), MERS-CoV (Middle East respiratory syndrome-related coronavirus), EriCoV (Hedgehog coronavirus), MHV (murine coronavirus), HCoV HKU1 (Human coronavirus HKU1), ChRCoV HKU24 (China Rattus coronavirus HKU24), ChRCovC HKU24 (Betacoronavirus 1), MrufCoV 2JL14 (Myodes coronavirus 2JL14), HCoV NL63 (Human coronavirus NL63), HCoV 229E (Human coronavirus 229E), and HCoV OC43 (Human coronavirus OC43). See, e.g., Coronaviridae Study Group of the International Committee on Taxonomy of Viruses, Nat Microbiol 2020. DOI: 10.1038/s41564-020-0695-z), which is specifically incorporated by reference in its entirety. In some embodiments, the coronavirus is a common cold coronavirus such as 229E, NL63, OC43, and HKU1.

In particularly preferred embodiments, the virus is a Severe acute respiratory syndrome-related virus, preferably one that infects humans such as SARS-CoV or SARS-CoV-2.

Various strains of the foregoing viruses are known and include the representative genomic sequences provided as, for example, the accession numbers provided herein, and those sequences and accession numbers provided in, e.g., Coronaviridae Study Group of the International Committee on Taxonomy of Viruses, Nat Microbiol 2020. DOI: 10.1038/s41564-020-0695-z. These, however, are non-limiting examples, and the disclosed methods can also be used to detect other strains of coronavirus, particularly SARS and MERS coronaviruses.

GenBank Accession No. MN908947.3, which is specifically incorporated by reference herein in its entirety, provides an exemplary genomic sequence (DNA) for SARS-CoV-2 (Severe acute respiratory syndrome coronavirus 2 isolate Wuhan-Hu-1, complete genome). Thus, in some embodiments, the (DNA sequence) of the viral genome has a sequence at least 80%, preferably at 85%, more preferably at least 86%, 87%, 88%, 89%, 90%, 91%, 92%, 93%, 94%, 95%, 96%, 97%, 98%, 99%, or 100% sequence identity to GenBank Accession No. MN908947.3 or a sequence or accession number provided in Coronaviridae Study Group of the International Committee on Taxonomy of Viruses, Nat Microbiol 2020. DOI: 10.1038/s41564-020-0695-z, all of which are specifically incorporated by reference herein in their entireties. It will be appreciated that the sequences are provided as DNA sequences, but the viral genome itself will typically have the corresponding RNA sequences. Thus, the corresponding RNA sequences are also expressly provided herein.

In some embodiments, the primers used in the disclosed compositions and methods (e.g., one-step RT-PCR) are designed against a target region in the nucleic acid sequence of GenBank Accession No. MN908947.3.

### IV. Kits

Also disclosed are kits for carrying out the disclosed methods. Compositions, reagents, and other materials can be packaged together in any suitable combination as a kit useful for performing, or aiding in the performance of, the disclosed methods. It is useful if the components of a given kit are designed and adapted for use together in the disclosed methods. For example, disclosed are kits with one or more primers, buffers, and/or enzymes. The kits may include a sterile needle, swab, syringe, ampule, tube, container, or other suitable vessels for isolating samples, holding assay components and/or performing the assay. The kits may include instructions for use.

The kit can include a sufficient quantity of reverse transcriptase (e.g., MMLV-RT), a DNA polymerase (e.g., Taq Pol), suitable nucleoside triphosphates, primers, and/or reaction buffer, or any combination thereof, for the amplification processes described above. A kit may further include instructions pertinent for the particular embodiment of the kit, such as providing conditions and steps for operation of the method. A kit may also contain reaction containers such as microcentrifuge tubes and the like. A kit may also reagents for isolating a biological sample and extracting nucleic acids therefrom.

The kits may contain nucleic acid primers suspended in an aqueous solution or as a freeze-dried or lyophilized powder, for instance. One or more control probes, primers, and or nucleic acids may be supplied in the kit. For example, the kit may include one or more positive control samples (such as a sample including a particular viral nucleic acid) and/or one or more negative control samples (such as a sample known to be negative for a particular viral nucleic acid).

In some embodiments, one or more primers (e.g., primers suitable for SARS-Cov-2), may be provided in pre-measured single use amounts in individual, typically disposable, tubes, wells, or equivalent containers. In this embodiment, the sample to be tested for the presence of the target nucleic acids can be added to the individual tube(s) or well(s) and amplification and/or detection can be carried out directly. In some examples, the containers may also contain additional reagents for amplification reactions, such as buffer, enzymes (such as reverse transcriptase and/or DNA polymerase), dNTPs, or other reagents. In some embodiments, the container includes all of the components required for the reaction except the sample (and water, if the reagents are supplied in dried or lyophilized form).

In some embodiments, the kit can contain reagents and instructions for detecting a viral nucleic acid. This can include for example, reagents, instructions, software and/or hardware for gel electrophoresis and data analysis.

The disclosed compositions and methods can be further understood through the following numbered paragraphs.
1. An RT-PCR reagent composition comprising a Moloney Murine Leukemia Virus Reverse Transcriptase (MMLV-RT) and a Thermus aquaticus DNA polymerase (Taq Pol) in effective amounts and in a buffer effective for one-step RT-PCR.
2. The composition of paragraph 1 comprising MMLV-RT and Taq Pol in a 2:1 ratio.
3. The composition of paragraph 2, wherein the ratio comprises nanograms of MMLV-RT to units (U) of Taq Pol.
4. The composition of any one of paragraphs 1-3 comprising from about 40 ng to about 80 ng MMLV-RT, from about 20 U to about 40 U Taq Pol, or combinations thereof.
5. The composition of paragraph 4 comprising about 60 ng MMLV-RT and about 30 U Taq Pol.
6. The composition of any one of paragraphs 1-5, wherein the MMLV-RT comprises the amino acid sequence of SEQ ID NO:1 or an amino acid sequence comprising at least 85% sequence identity to SEQ ID NO:1.
7. The composition of any one of paragraphs 1-6, wherein the Taq Pol comprises the amino acid sequence of SEQ ID NO:2 or an amino acid sequence comprising at least 85% sequence identity to SEQ ID NO:2.
8. The composition of paragraph 6 or 7, wherein the MMLV-RT and/or Taq Pol further comprise one or more tag sequences at the N-terminus and/or C-terminus.
9. The composition of paragraph 8, where the tag sequence is selected from His tag and Strep tag.
10. The composition of any one of paragraphs 1-9, wherein the buffer comprises one or more of a salts a reducing agent, a chelating agent, a detergent a buffering agent, a plurality of deoxynucleoside triphosphates (dNTPs), or combinations thereof.
11. The composition of paragraph 10, wherein the one or more salts are selected from the group comprising KCl, MgCl₂, and (NH₄)₂SO₄.
12. The composition of paragraph 10 or 11, wherein the dNTPs are selected from the group consisting of dATP, dTTP, dGTP, and dCTP.
13. The composition of any one of paragraphs 10-12 comprising 20-50 mM Tris-HCl (pH 8.5); 75-150 mM KCl; 2-4 mM MgCl₂; 0.5-2 mM DTT; 200-500 μM dNTPs and 13.5 mM (NH₄)₂SO₄.
14. A method of performing a one-step RT-PCR comprising forming a mixture by combining an RNA sample/template and a plurality of primers with the composition of any one of paragraphs 1-13 and incubating the mixture under conditions sufficient to amplify one or more DNA molecules complementary to one or more portions of the RNA sample/template.
15. A method of detecting presence of a nucleic acid in a sample comprising combining the sample and plurality of primers specific to the nucleic acid with the composition of any one of paragraphs 1-13 under conditions sufficient for amplification of the nucleic acid, and detecting the nucleic acid amplification product, thereby detecting presence of the nucleic acid in the sample.
16. The method of paragraph 15, wherein the nucleic acid is derived from a coronavirus, preferably a severe acute respiratory syndrome-related coronavirus, more preferably SARS-CoV-2.
17. The method of any one of paragraphs 14-16, wherein the sample is an RNA sample derived from mucus, sputum (processed or unprocessed), saliva, bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.
18. A method of diagnosing a subject for infection with a virus comprising detecting the presence of a viral nucleic acid in a sample from the subject by the method of any one of paragraphs 15-17, wherein detecting the amplification product indicates the subjects is infected with the virus.
19. The method of paragraph 18, wherein the subject exhibits or does not exhibit symptoms of a disease, disorder, or condition associated with the virus.
20. The method of paragraph 18 further comprising treating the subject, wherein the subject was diagnosed as infected with the virus.
21. The method of paragraphs 18-20, wherein the subject is human.
22. The composition of any one of paragraphs 1-13 wherein the MMLV-RT sequence comprises a sequence represented by SSAG-A-L₁-B-L₂-C, where: (i) SSAG is an amino acid sequence or a variant thereof made by conservative substitution of the amino acids therein; (ii) A is a peptide cleave sequence, (iii) B is a protein tag as disclosed herein, (iv) L₁ and L₂ are optional first linkers, and (v) B is a second protein tag.
23. The composition of any one of paragraphs 1-13, wherein the MMLV-RT comprises SSAGENLYFQGSSSHHHHHHHHGGGSAWSHPQFEK (SEQ ID NO: 20).

### EXAMPLES

### Example 1: Development of a One-Step Taq Pol and MMLV-RT Based RT-qPCR Kit Useful for Disease Diagnosis

### Materials and Methods

Expression and Purification of Taq Pol

The full-length gene of Taq Pol in pENTR-Taq vector was transferred to an in house pColdDest vector using Gateway LR reaction (Thermofisher). The resulting plasmid was termed pColdDest-His-Taq (FIG. 2B). The expression plasmid of His-Taq Pol was transformed into BL21(DE3) E. coli strain and cells were grown in 10 L LB medium to an OD₆₀₀ of 0.8. The overexpression of His-Taq Pol was induced by 1.0 mM Isopropyl β-D-1-thiogalactopyranoside (IPTG) at 16° C. for 16 hours. The cells were then harvested by centrifugation at 5,500×g for 15 minutes, re-suspended in Buffer A [50 mM Tris-HCl (pH 7.5), 0.5 M NaCl, 1 mM DTT, 10% (v/v) Glycerol, 0.5% NP-40], and incubated on ice with Lysozyme at 1 mg/mL final concentration for 60 minutes. The cells were disrupted by two cycles of sonication (35% amplitude, 10 second on/off cycle for 5 minutes). Cell debris was removed by centrifugation at 22,040×g for 30 minutes and the clear supernatant was collected and incubated at 75° C. for 15 minutes to denature the endogenous proteins from E. coli. The heat-denatured solution was then cooled down quickly on ice and centrifuged at 96,000×g for 45 minutes to remove the denatured proteins. The decanted supernatant was filtered through a 0.45 μM pore size filter and directly loaded onto His-Trap HP 5 mL (GE Healthcare) column pre-equilibrated with Buffer B [20 mM Tris-HCl (pH 7.5), 0.5 M NaCl]+20 mM Imidazole. The column was then washed with 20 column volumes (CVs) of Buffer B. The proteins were eluted by 20 CV gradient against Buffer B+500 mM Imidazole. The peak fractions were analyzed by SDS-PAGE, and the His-Taq Pol containing-fractions were pooled and dialyzed overnight in Buffer C [20 mM Tris-HCl (pH 7.5), 50 mM NaCl, 1 mM EDTA]. The dialyzed sample was then loaded onto HiTrap SP 5 mL (GE Healthcare) column pre-equilibrated with Buffer C. The proteins were eluted by 20 CV gradient against Buffer D [20 mM Tris-HCl (pH7.5), 1 mM EDTA, 1 M NaCl]. The peak fractions were analyzed by SDS-PAGE, pooled, and the fractions carrying pure His-Taq Pol were dialyzed against Buffer E [50 mM Tris-HCl (pH 8.0), 25 mM NaCl, 0.1 mM EDTA, 1 mM DTT, 0.5% Tween-20, 0.5% NP-40, 50% (v/v) Glycerol]. The dialyzed samples were stored in −20° C.

Expression and Purification of MMLV-RT

The full-length gene of MMLV-RT with cleavable TEV-8xHis-Strep tag at the C-terminus was cloned into the pDEST8 expression plasmid as previously described [52]. The plasmid was designated as pDEST8-MMLVRT-TEV-His8-Strept. The C-His/Strep MMLV-RT (hereafter named MMLV-RT) expression plasmid was then transformed into DH10Bac cells (Life Technologies) to prepare the bacmid DNA for the transfection of Sf9 insect cells. The Sf9 cells were cultured in ESF 921 medium (Expression Systems) at 27° C. with continuous shaking at 80 rpm for aeration. To prepare the baculovirus, the MMLV-RT bacmaid DNA was subsequently transfected into Sf9 cells using FuGENE® HD reagent (Promega) per manufacturer's instructions. The resulting supernatant was collected as P1 virus stock then amplified to obtain P2 virus stock, which was further amplified to generate P3 virus stock for large-scale expression. The expression of MMLV-RT then proceeded by transfecting 8 L of Sf9 suspension culture at a density of 2×10⁶ cells/nL with P3 virus. 55 hours post transfection with P3 virus, the cells were harvested by centrifugation at 5,500×g for 10 minutes. The cell pellet was re-suspended in 3 mL per 1 g of wet cells in Buffer F [50 mM Tris-HCl (pH 8.0), 300 mM NaCl, 0.1% NP-40, 1 mM PMSF, 1 mM EDTA, 5% (v/v) Glycerol, and EDTA-free protease inhibitor cocktail tablets (Roche, UK) at 1 tablet per 50 mL lysis buffer]. All the later steps were performed at 4° C., where the suspended cells were sonicated and debris was removed by centrifugation at 95,834×g for 1 hour at 4° C.

The clear lysate was directly loaded onto HisTrap Excel 5 mL affinity column (GE Healthcare) pre-equilibrated with Buffer G [50 mM Tris-HC (pH 8.0), 300 mM NaCl, 0.1% NP-40, 1 mM EDTA, 5% (v/v) Glycerol]. The column was then washed with 10 CVs of Buffer G followed by washing with another 10 CVs of Buffer G+25 mM Imidazole. Finally, the bound proteins were eluted by 10 CV gradient against Buffer G+500 mM Imidazole. The peak fractions were pooled and dialyzed overnight against Buffer H [100 mM Tris-HCl (pH 8.0), 150 mM NaCl, 1 mM EDTA and 5% (v/v) Glycerol]. The dialyzed sample was then loaded onto StrepTrap XT 5 mL (GE Healthcare) pre-equilibrated with Buffer H. The column was then washed with 10 CVs of Buffer H and eluted by 10 CVs of Buffer H+50 mM biotin. Fractions containing MMLV-RT were pooled and dialyzed overnight against storage Buffer I [20 mM Tris-HCl (pH 7.5), 100 mM NaCl, 0.01% NP-40, 0.1 mM EDT A, 50% (v/v) Glycerol], then flash frozen in liquid nitrogen and stored at −80° C. The protein concentration was determined by measuring the absorbance at 280 nm using an extinction coefficient calculated based on the amino acid sequence of the protein.

PCR Reaction

The PCR reactions by Taq Pol were performed in reaction buffer [20 mM Tris-HCl (pH 8.4), 50 mM KCl, 1.5 mM MgCl₂, 200 μM dNTPs] besides various amounts of ammonium sulfate and MMLV-RT. pUC19 vector (Invitrogen) was used as a template to amplify the ampicillin resistance gene with the following primers: 5′-TTACCAATGCTTAATCAGTGAGGCACC-3′ (SEQ ID NO:28) and 5′-ATGAGTATTCAACATTTCCGTGTCGCCC-3′ (SEQ ID NO:29). The heating and cooling program used for the PCR reactions involves; pre-denaturation for 2 minutes at 94° C. followed by cycling composed of the three steps: denaturation for 15 seconds, annealing for 15 seconds at 94° C., and extension for 1 minute at 68° C. The cycle is then repeated for 29 times.

Determination of the Unit of Taq Pol's Activity

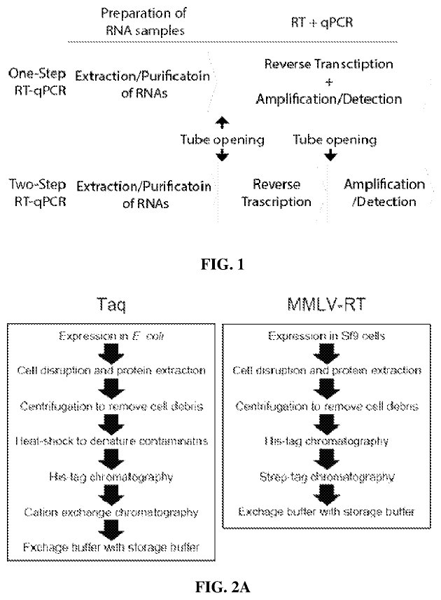

The unit of Taq Pol's activity was determined based on the standard titration curve of native Taq Pol's activity (Thermofisher) (FIG. 1G). Serial dilutions of native Taq Pol (5 U, 2.5 U, 1 U, 0.5 U, and 0.25 U) were used for PCR reactions. The PCR products were analyzed on 1% agarose gel, and the gel images were captured by iBright Imaging System (Thermofisher). The intensities of the bands corresponding to 862 bp, which is the gene size of ampicillin resistance in pUC19, were quantified by iBright Analysis Software (Thermofisher). The unit of His-Taq Pol's activity was determined by interpolation from the standard curve using Prism8 software (GraphPad).

Reverse Transcriptase Activity Assay

The reaction mixture containing 1 μL of RNA template (10⁶ copies/μL of SARS-CoV-2 N gene RNA), 2 μL of random hexamers (100 ng), 1 μL of dNTPs (0.5 mM), and 4 μL of dH₂O was heated to 65° C. for 5 minutes followed by immediate cooling by placing on ice for more than 1 minute. After the sample tubes were centrifuged briefly, 4 μL of 5×RT buffer, 4 μL of dH₂O, 2 μL of DTT (10 mM), and 1 μL of RNaseOUT (40 units) (Invitrogen Cat No 10777019) were added and further incubated at room temperature for 2 minutes. Then 1 μL of enzymes was added to the respective reaction of either MMLV-RT, ProtoScript® II (New England Labs Cat No M0368), NEB AMV (New England Labs Cat No M0277), Superscript II (Invitrogen Cat. No 18064022), or Superscript III (Invitrogen Cat. No 18080093). The final reaction mixtures were incubated at room temperature for 10 minutes, followed by incubation at 42° C. for 50 minutes then by heat inactivation at 70° C. for 15 minutes. Lastly, 5 units of thermostable RNase H (New England Biolabs Cat. No M0523S) were added and incubated at 37° C. for 20 minutes to hydrolyze RNA. The resulting cDNAs were subjected to RT-PCR assays with 2019-nCoV_N3 primers (IDT Cat. No 10006605) (Table 1). The RT-PCR reactions were performed on CFX384 Touch Real-Time PCR Detection System (BioRad) using IQ Multiplex Powermix (BioRaD Cat. No 172-5849) and the following program; pre-denaturation at 95° C. for 2 minutes followed by 45 cycles of denaturation at 95° C. for 5 seconds and by annealing/extension at 59° C. for 30 seconds.

Primer and Probe Sets

The CDC designed RT-qPCR assay primers and probes set (2019-nCoV CDC EUA kit, catalogue no. 10006606) was purchased from Integrated DNA Technologies (IDT). The kit contains research-use-only primer and probe sets based on the protocol released by CDC (hereafter called CDC assay). The CDC assay includes three sets of the primers and probe labeled with 5′ FAM dye and 3′ Black Hole Quencher® (BHQ) (Table 1).

TABLE 1
Sequences of primers and probes used in the CDC assays

| Primer | | Oligonucleotide Sequence | |
|---|---|---|---|
| Name | Description | (5′-3′) | Modification |
| N1-F | 2019-nCoV-N1 | 5′-GACCCCAAAATCAGCGAAAT-3′ | None |
| | Forward Primer | (SEQ ID NO: 3) | |
| N1-R | 2019-nCOV-N1 | 5′- | None |
| | Reverse Primer | TCTGGTTACTGCCAGTTGAATCTG- | |
| | | 3′ (SEQ ID NO: 4) | |
| N1-P | 2019-nCOV-N1 | 5′-FAM- | FAM/BHQ1 |
| | Probe | ACCCCGCATTACGTTTGGTGGACC- | |
| | | BHQ1-3′(SEQ ID NO: 5) | |
| N2-F | 2019-nCoV-N2 | 5′-TTACAAACATTGGCCGCAAA-3′ | None |
| | Forward Primer | (SEQ ID NO: 6) | |
| N2-R | 2019-nCOV-N2 | 5′-GCGCGACATTCCGAAGAA-3′ | None |
| | Reverse Primer | (SEQ ID NO: 7) | |
| N2-P | 2019-nCOV-N2 | 5′-FAM- | FAM/BHQ1 |
| | Probe | ACAATTTGCCCCCAGCGCTTCAG- | |
| | | BHQ1-3′ (SEQ ID NO: 8) | |
| N3-F | 2019-nCoV-N3 | 5′-GGGAGCCTTGAATACACCAAAA- | None |
| | Forward Primer | 3′ (SEQ ID NO: 9) | |
| N3-R | 2019-nCOV-N3 | 5′-TGTAGCACGATTGCAGCATTG-3′ | None |
| | Reverse Primer | (SEQ ID NO: 10) | |
| N3-P | 2019-nCOV-N3 | 5′-FAM- | FAM/BHQ1 |
| | Probe | AYCACATTGGCACCCGCAATCCTG- | |
| | | BHQ1-3′ (SEQ ID NO: 11) | |
| RP-F | RNAse P | 5′-AGATTTGGACCTGCGAGCG-3′ | None |
| | Forward Primer | (SEQ ID NO: 12) | |
| RP-R | RNAse P | 5′-GAGCGGCTGTCTCCACAAGT-3′ | None |
| | Reverse Primer | (SEQ ID NO: 13) | |
| RP-P | RNAse P | 5′-FAM- | FAM/BHQ1 |
| | Probe | TTCTGACCTGAAGGCTCTGCGCG- | |
| | | BHQ1-3′ (SEQ ID NO: 14) | |

DNA/RNA Positive Controls

The 2019-nCoV-N-Positive Control (IDT, catalog no. 10006625) is composed of plasmids containing the complete N gene (1,260 base pairs) of SARS-CoV-2. The Hs-RPP30 Positive Control (IDT, catalog no. 10006626) contains a portion of the ribonuclease P30 subunit (RPP30) gene of the human genome. The control SARS-CoV-2 viral RNA sequences used for constructing RNA titration curves were synthetic RNAs from six sequence variants of the SARS-CoV-2 virus (Twist Bioscience). The dried stock was re-suspended in 100 μL of 1×TE Buffer [110 mMv Tris-HCl and 1 mMv EDTA (pH 8.0)] to make a stock of 1×10⁶ RNA copies/μL.

Clinical Specimen and RNA Extraction

Nasopharyngeal swabs were collected from COVID-19 suspected patients in Ministry of Health hospital in the Western region in Kingdom of Saudi Arabia. The swabs were placed in 2 mL screw capped cryotubes containing 1 mL of TRIzol (Ambion) for inactivation and transport to King Abdullah University of Science and Technology (KAUST) for further downstream applications. The sample tubes were sprayed with 70% ethanol, and RNAs were extracted within 2 hours using the Direct-zol RNA Miniprep kit (Zymo Research) per the manufacturer's instructions, along with several optimizations to improve the quality and quantity of the extracted RNAs from clinical samples. The optimization also included extending the TRIzol incubation period and the addition of chloroform during the initial lysis step to obtain the aqueous RNA layer. The quality control of purified RNAs was performed using the High Sensitivity Qubit kit (Thermo Fisher) and RNA 6000 Nano Agilent kit (Agilent), respectively.

One-step RT-qPCR

To determine the sensitivity of the disclosed one-step RT-qPCR kit and to detect SARS-CoV-2 in clinical samples, RT-qPCR was performed using primers and probes from 2019-nCOV CDC EUA Kit produced by IDT (Catalogue Number: 10006606). This kit contains three sets of two primers and one probe; two sets for nucleocapsid gene (N1 and N2) of the viral genome and one set for human RNase P gene, following the CDC diagnostic panel (https://www.fda.gov/media/134922/download). Briefly, the reaction mixture included 10 μL of 2× reaction buffer mix, 1 L of His-Taq Pol/C-His/Strep MMLV-RT enzyme mix, 1 L of probe/primer mix, 1 or 2 L of RNA and nuclease-free water to reach a total of 20 μL reaction volume. RT-qPCR was performed in ABI 7900 Fast Real-Time PCR system (Applied Biosystems, USA). The RT-qPCR conditions were as follows: reverse transcription at 55° C. for 30 minutes, pre-denaturation at 94° C. for 2 minutes followed by cycling composed of the three steps: denaturation at 94° C. for 15 seconds, by annealing at 58° C. for 30 seconds, and by extension at 68° C. for 1 minute repeated for 45 cycles. At the end, the reaction was heated at 68° C. for 5 minutes.

### Results

Expression and Purification of Histidine-Tagged Taq Polymerase

The expression and purification of the native form of Taq Pol was established in 1991 [53]. To make both expression and purification processes straightforward, Taq Pol with N-terminal histidine-tag was expressed under the control of the cold-shock promoter. The construct layout of the expression vector for His-Taq Pol is depicted in FIG. 2B. The procedures for the expression and purification of His-Taq Pol are outlined in FIG. 2A and described in detail above. With this new construct, the induction and expression level can be precisely controlled by adjusting IPTG concentration and varying the temperature. As shown in FIG. 2C, a noticeable overexpression of His-Taq Pol in comparison to the endogenous E. coli proteins was observed by incubating at 16° C. with 1 mM IPTG final concentration. In contrast, such enhancement was not detected for the expression of native Taq Pol (N-Taq Pol) under the control of pET vector system (data not shown). Tagging at the N-terminus with histidines also allowed for elimination of the time-consuming polyethylenimine precipitation steps in the previous purification protocol [53] without affecting the purity of the final product. As shown in FIG. 2C, the purity of the His-Taq Pol was comparable to that of the commercially available N-Taq Pol. Owing to the improved expression level of Taq Pol, as illustrated by the final yield of the purified protein from 1 L E. coli culture (FIG. 2E), the 0.98 mg of pure protein was obtained equivalent to ˜$105,000 in market value.

Expression and Purification of Double his- and Strep-Tagged MMLV-RT

Next, a C-terminal double His- and Strep-tagged MMLV-RT (C-His/Streo MMLV-RT) protein was expressed in insect cells (Sf9) using the baculovirus expression system (FIG. 2B). The expression and purification protocols are outlined in FIG. 2A and described in detail above. Previous studies demonstrated that C-His/Strep MMLV-RT exhibits higher activity compared to the N-terminal tagged one (N-His/Strep MMLV-RT), where both proteins were produced in the silkworms using the silkworm-baculovirus expression vector system (silkworm-BEVS) [52]. Therefore, further experiments opted for the expression and purification of the C-His/Strep MMLV-RT from Sf9 cell suspension culture in order to obtain homogenous proteins by implementing the previously established protocol [52]. The final products were assessed to be more than 95% pure (FIG. 2D). Remarkably, the closing yields of the purified proteins are around 7.5 mg per 1 L insect cell culture, i.e. 7.6 times more than that of His-Taq Pol expressed in E. coli. These results suggest that the C-terminus Histidine and Strep double-affinity tagged system enhanced the expression of C-His/Strep MMLV-RT in Sf9 cells. Moreover, two consecutive steps of affinity chromatography resulted in minimal loss of the proteins during the purification. Since both the native form and C-His/Strep MMLV-RT showed the same RT activity [52], the cleavage step of the TEV tag was omitted in favor of achieving higher final yield of C-His/Strep MMLV-RT.

### Activity Assays of Taq Pol and MMLV-RT

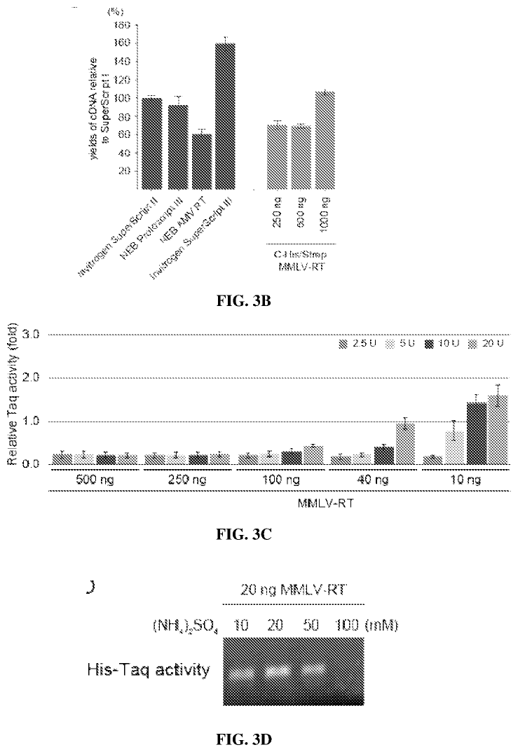

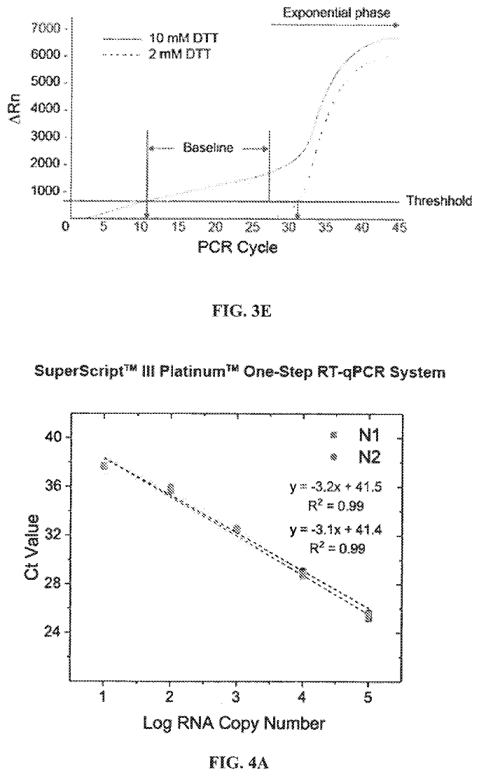

In order to evaluate the activities of both His-Taq Pol and C-His/Strep MMLV-RT, PCR reactions were performed for His-Taq Pol and the reverse transcriptase assay for C-His/Strep MMLV-RT (FIGS. 3A and 3B, respectively). The relatively high concentration of detergents, i.e. 0.5% NP-40 and Tween-20, in the storage buffer of Taq Pol hindered the accurate determination of its concentration based on UV absorbance measurement due to the high background. Therefore, the protein amount was determined by measuring its PCR amplification activity relative to commercial N-Taq Pol (Invitrogen). The optimal dilution factor was determined by the storage buffer for purified His-Taq Pol to be 160 times. The dilution circumvents the inhibitory effect of high concentration of His-Taq Pol on its own PCR amplification activity (FIG. 3A). A standard titration curve of the activity of N-Taq Pol versus enzyme unit was also constructed, to define and calculate the unit of the purified His-Taq Pol's catalytic activity (310 units/μL).

The activity of the purified C-His/Strep MMLV-RT was assessed using two-step RT-qPCR in comparison to various commercially-available reverse transcriptases (FIG. 3B). The activity of a fixed amount of 200 units containing approximately 1,000 ng enzyme from each commercial reverse transcriptase preparation was compared to that of variable quantities of C-His/Strep MMLV-RT. The data showed that the activity of the C-His/Strep MMLV-RT within the range of 250-1,000 ng was almost consistent to SuperScript II, NEB Protoscript II, and NEB AMV-RT. Collectively, these studies confirmed that the disclosed purified His-Taq Pol and C-His/Strep MMLV-RT are competent for the PCR and reverse transcription reactions. Hereon, amounts of the purified His-Taq Pol is quantified in units and amounts of C-His/Strep MMLV-RT in nanograms (ng).

Inhibitory Effect of MMLV-RT on Taq Pol

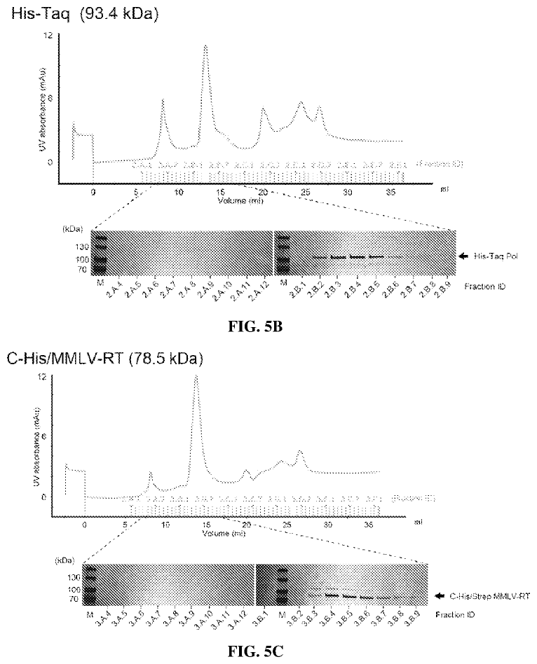

To perform two distinct reactions within the same tube, the two enzymes, Taq Pol and MMLV-RT, should work simultaneously, collaboratively, or at least independently without inhibiting one another. However, it was reported by Sellner et al. that MMLV-RT inhibits the activity of Taq Pol in the PCR reaction [51]. To assess the magnitude of the inhibitory effect of MMLV-RT on the Taq Pol's activity, PCR reactions were performed at varying portions of C-His/Strep MMLV-RT relative to His-Taq Pol (FIG. 3C). Per manufacturer's instructions, the recommended quantity of the reverse transcriptase to synthesize cDNA from total RNAs with dT or random hexamer primers is 200 units (1 μg). Therefore, 500 ng of C-His/Strep MMLV-RT was used in combination with the range of 2.5-20 units of His-Taq Pol and gradually decreased the amount of C-His/Strep MMLV-RT in the reaction. The results showed C-His/Strep MMLV-RT at 100 ng or more completely inhibits the Taq Pol's activity even at 20 units of His-Taq Pol. However, by reducing the amount of C-His/Strep MMLV-RT to 40 ng, its inhibitory effect could be overcome by using 20 units of His-Taq Pol. SDS-PAGE analysis of the samples after the reaction showed that the two enzymes remained separate with no apparent cross-linking interaction (FIG. 2F). Furthermore, using size exclusion chromatography (SEC) analysis to evaluate the physical interaction between His-Taq Pol and C-His/Strep MMLV-RT, no molecular-size shift was detected in the presence of the two enzymes (FIG. 5A-C). These results suggest that MMLV-RT hampers the Taq Pol's activity with a mechanism other than physical interaction. In conclusion, to overcome the inhibitory effect of MMLV-RT on the Taq Pol's activity, the present studies determined that the maximum tolerable amount of MMLV-RT is 40 ng in the presence of 20 units of Taq Pol. Therefore, the mixing ratio to be 2 ng MMLV-RT to 1 unit Taq Pol was selected for subsequent buffer component selection experiments.

Selecting Buffer Components to Support One-Step RT-qPCR Reaction

The salt composition of the buffer was investigated to further improve the Taq Pol's activity in the PCR reaction with MMLV-RT. Ammonium sulfate was selected as additional salt and different concentrations were tested in the PCR reactions (FIG. 3D). The results showed that the intensity of the PCR product bands gradually increased up to 20 mM of ammonium sulfate followed by decrease at higher concentration of ammonium sulfate due to the suppression of the His-Taq Pol's activity. Another important parameter for the determination of the optimal buffer composition for the one-step RT-qPCR reaction is DTT concentration. Typically, a relatively high concentration, 10 mM, of DTT is used in the reverse transcription reaction. The effect of DTT concentration in the context of the one-step RT-qPCR reactions was investigated (FIG. 3E). At 1 mM DTT, the baseline remained close to zero before the PCR amplification entered into the exponential phase, whereas at 10 mM DTT a gradual increase in the baseline was observed. This baseline drift might be attributed to the degradation of the probe resulting in a higher noise level. With the further optimizations of the one-step RT-qPCR reactions, the following favorable buffer composition was selected for the subsequent one-step RT-qPCR assays [50 mM Tris-HCl pH (8.5), 75 mM KCl, 2 mM MgCl₂, 1 mM DTT, 200 μM dNTPs and 13.5 mM ammonium sulfate].

Determination of the Enzyme Amounts Used in the One-Step RT-qPCR Reaction, its Sensitivity and SARS-CoV-2 Detection Limit

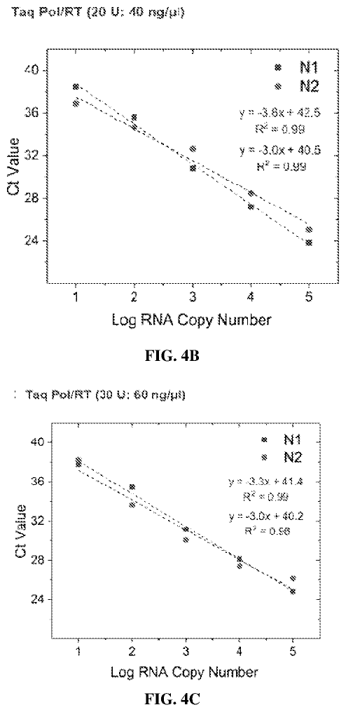

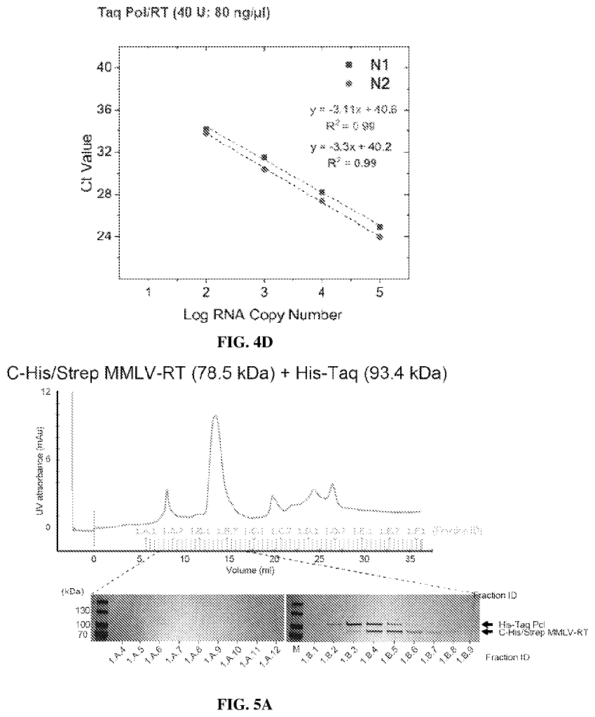

In order to successfully develop a one-step RT-qPCR platform for the detection of SARS-CoV-2, purified Taq Pol and MMLV-RT were formulated and their efficiency in the one-step RT-qPCR scheme was evaluated using N1 and N2 primer sets and synthetic SARS-CoV-2 RNA as a template. The data showed that a mixing ratio of 2 ng C-His/Strep MMLV-RT to 1 unit His-Taq Pol still sustained the Taq Pol's activity in PCR (FIG. 3C). Therefore, in initial screenings, the optimal amount of MMLV-RT in the RT-qPCR reaction while keeping its relative ratio to Taq Pol within this acceptable activity range was determined. Three combinations at diverse His-Taq Pol and C-His/Strep MMLV-RT ratios; 20 U: 40 ng/μL, 30 U: 60 ng/μL, and 40 U: 80 ng/μL were used in each reaction and quantitatively compared to Invitrogen SuperScript™ III Platinum™ One-Step RT-qPCR System (Catalogue Number: 12574026). Three independent replicas of the RT-qPCR were performed using N1 and N2 primer sets and applying 10-fold serial dilutions of synthetic SARS-CoV-2 RNA ranging from 10 to 105 copies/μL as a template. The sensitivity of the one-step RT-qPCR system was estimated in terms of the limit of detection (LoD). The LoD assays demonstrated that one-step RT-qPCR system reliably detected 40 RNA copies per reaction in all of the three aforementioned combinations (data not shown). Furthermore, the present data illustrated that the one-step RT-qPCR assays with 30 U: 60 ng/μL ratio was able to detect as low as 10 RNA copies per reaction from 9 out of 10 replicates as compared to 7 out of 10 replicates in case of 20 U: 40 ng/μL and 3 out of 10 replicates in case of 40 U: 80 ng/μL ratios, indicating the higher sensitivity of the system upon using 30 U: 60 ng/μL ratio. The slope and the R² values of each curve were used to evaluate the efficiency of individual assays. The R² values provided an estimate of the goodness of the linear fit to the data points. In an efficient qPCR assay, R² should be very close or greater than 0.90. The amplification efficiencies of all three His-Taq Pol/C-His/Strep MMLV-RT ratios were above 99% for both N1 and N2 primer sets (FIGS. 4B, 4C, and 4D). The standard curves showed high correlation coefficients of R²>0.99 for N1 primer set and R²>0.95 for N2 with the three His-Taq Pol/C-His/Strep MMLV-RT ratios.

Validation of the One-Step RT-qPCR System for the Detection of SARS-CoV-2 in Clinical Samples

In order to validate the competency of the one-step RT-qPCR system to detect SARS-CoV-2 in clinical samples from patients, RNA samples extracted from the nasopharyngeal swabs of 20 different patients who tested positive besides three patients who tested negative for SARS-CoV-2 using RT-qPCR assays were used (Table 2). The positive samples had variable Ct values ranging from 16 to 38 applying the CDC qPCR N gene primer set (IDT, catalogue no. 10006606). Based on LoD results, 30 U: 60 ng/μL ratio was used to evaluate the performance of the one-step RT-qPCR system on actual clinical samples. The internal control human RNAseP gene (RP) was detected in all samples. As expected, the N1 and N2 primer pair of the viral N-gene was only detected in the positive samples not the negative ones (Table 2). In case of the three negative control samples (411, 429 and 440) Invitrogen SuperScript™ III Platinum™ One-Step RT-qPCR System gave some high Ct values upon applying N1 primer set but overall result undetermined. The overall Ct values demonstrated a narrow difference between the one-step RT-qPCR system and Invitrogen SuperScript™ III Platinum™ One-Step RT-qPCR System (Table 2) demonstrating the sensitivity of the for the detection of SARS-CoV-2 from clinical samples.

TABLE 2
Comparison of One-step RT-qPCR System with
Invitrogen SuperScript ™ III Platinum ™ One-
Step RT-qPCR System

| | | | | | | |
|---|---|---|---|---|---|---|
| Sr. No. | Sample | N1 | N2 | RP | Expected | Result |

One-step RT-qPCR System

| | | | | | | |
|---|---|---|---|---|---|---|
| 1 | 613 | 16.9 | 17.5 | 27.3 | + | Positive |
| | | | | | | SARS CoV-2 |
| 2 | 632 | 17.8 | 18.6 | 27.9 | + | Positive |
| | | | | | | SARS CoV-2 |
| 3 | 581 | 20.5 | 21.8 | 25.1 | + | Positive |
| | | | | | | SARS CoV-2 |
| 4 | 599 | 22.1 | 23.6 | 23.9 | + | Positive |
| | | | | | | SARS CoV-2 |
| 5 | 583 | 24.5 | 25.7 | 25.8 | + | Positive |
| | | | | | | SARS CoV-2 |
| 6 | 572 | 27.0 | 28.3 | 24.6 | + | Positive |
| | | | | | | SARS CoV-2 |
| 7 | 562 | 30.0 | 30.6 | 28.4 | + | Positive |
| | | | | | | SARS CoV-2 |
| 8 | 291 | 26.9 | 29.0 | 23.7 | + | Positive |
| | | | | | | SARS CoV-2 |
| 9 | 380 | 27.1 | 27.4 | 23.5 | + | Positive |
| | | | | | | SARS CoV-2 |
| 10 | 550 | 32.8 | 33.1 | 26.9 | + | Positive |
| | | | | | | SARS CoV-2 |
| 11 | 552 | 32.6 | 35.4 | 30.7 | + | Positive |
| | | | | | | SARS CoV-2 |
| 12 | 554 | 33.9 | 35.0 | 31.9 | + | Positive |
| | | | | | | SARS CoV-2 |
| 13 | 585 | 35.5 | 38.4 | 24.1 | + | Positive |
| | | | | | | SARS CoV-2 |
| 14 | 600 | 35.3 | 38.3 | 26.2 | + | Positive |
| | | | | | | SARS CoV-2 |
| 15 | 601 | 34.6 | 36.7 | 23.4 | + | Positive |
| | | | | | | SARS CoV-2 |
| 16 | 745 | 34.0 | 37.0 | 28.1 | + | Positive |
| | | | | | | SARS CoV-2 |
| 17 | 748 | 31.1 | 33.4 | 27.0 | + | Positive |
| | | | | | | SARS CoV-2 |
| 18 | 749 | 34.3 | 36.1 | 32.9 | + | Positive |
| | | | | | | SARS CoV-2 |
| 19 | 751 | 35.0 | 37.2 | 29.6 | + | Positive |
| | | | | | | SARS CoV-2 |
| 20 | 750 | 34.5 | 38.3 | 37.6 | + | Positive |
| | | | | | | SARS CoV-2 |
| 21 | 411 | — | — | 27.5 | − | Not Detected |
| 22 | 429 | — | — | 26.9 | − | Not Detected |
| 23 | 440 | — | — | 25.1 | − | Not Detected |
| 24 | + | 24.8 | 25.1 | 24.8 | − | Not Detected |
| 25 | NTC | — | — | — | − | Not Detected |

Invitrogen SuperScriptTM III PlatinumTM One-Step RT-qPCR System

| | | | | | | |
|---|---|---|---|---|---|---|
| 1 | 613 | 17.5 | 17.3 | 27.2 | + | Positive |
| | | | | | | SARS CoV-2 |
| 2 | 632 | 18.5 | 18.6 | 27.9 | + | Positive |
| | | | | | | SARS CoV-2 |
| 3 | 581 | 21.6 | 22.1 | 25.3 | + | Positive |
| | | | | | | SARS CoV-2 |
| 4 | 599 | 23.2 | 23.7 | 23.9 | + | Positive |
| | | | | | | SARS CoV-2 |
| 5 | 583 | 25.9 | 26.1 | 26.3 | + | Positive |
| | | | | | | SARS CoV-2 |
| 6 | 572 | 28.5 | 29.0 | 28.8 | + | Positive |
| | | | | | | SARS CoV-2 |
| 7 | 562 | 29.5 | 30.0 | 27.3 | + | Positive |
| | | | | | | SARS CoV-2 |
| 8 | 291 | 29.8 | 31.3 | 24.5 | + | Positive |
| | | | | | | SARS CoV-2 |
| 9 | 380 | 26.8 | 27.1 | 24.4 | + | Positive |
| | | | | | | SARS CoV-2 |
| 10 | 550 | 33.0 | 33.9 | 26.5 | + | Positive |
| | | | | | | SARS CoV-2 |
| 11 | 552 | 34.2 | 34.7 | 30.2 | + | Positive |
| | | | | | | SARS CoV-2 |
| 12 | 554 | 33.2 | 33.4 | 28.6 | + | Positive |
| | | | | | | SARS CoV-2 |
| 13 | 585 | 35.4 | 37.6 | 23.8 | + | Positive |
| | | | | | | SARS CoV-2 |
| 14 | 600 | 35.9 | 37.2 | 26.3 | + | Positive |
| | | | | | | SARS CoV-2 |
| 15 | 601 | 35.2 | 36.1 | 23.0 | + | Positive |
| | | | | | | SARS CoV-2 |
| 16 | 745 | 35.3 | 36.7 | 28.1 | + | Positive |
| | | | | | | SARS CoV-2 |
| 17 | 748 | 32.1 | 34.1 | 26.9 | + | Positive |
| | | | | | | SARS CoV-2 |
| 18 | 749 | 34.1 | 34.7 | 32.5 | + | Positive |
| | | | | | | SARS CoV-2 |
| 19 | 751 | 34.9 | 36.1 | 28.6 | + | Positive |
| | | | | | | SARS CoV-2 |
| 20 | 750 | 35.2 | 36.8 | 36.7 | + | Positive |
| | | | | | | SARS CoV-2 |
| 21 | 411 | 36.9 | — | 29.1 | − | Not Detected |
| 22 | 429 | 38.3 | 40.5 | 27.5 | − | Not Detected |
| 23 | 440 | 37.6 | — | 25.5 | − | Not Detected |
| 24 | + | 23.6 | 23.6 | 24.2 | − | Not Detected |
| 25 | NTC | — | — | — | − | Not Detected |

### Discussion

SARS-CoV-2 is the etiological agent of the present international outbreak of a severe respiratory syndrome termed as COVID-19. The outburst of this viral infection has become a pandemic and severe public health challenge worldwide. Owing to the absence of effective medicines and vaccines, quick identification of the infected individuals and imposing self-quarantine measurements are the only effective containment strategies to avoid widespread community transmission. Therefore, the rapid development of low-cost, easy-to-make, yet sensitive and reliable diagnostic tests are crucial.

In this study, a sensitive, cost-effective, in-house one-step RT-qPCR system based on Taq Pol and MMLV-RT was devised. These enzymes were discovered in the 1970s [54, 55], although their application in one-step-PCR has not been demonstrated. The experiments led to successful purification of these enzymes and selection of reaction conditions allowing for their use for the one-step RT-qPCR platform. To simplify the expression and purification procedures, His-Taq Pol was used under the control of the cold-shock promoter for E. coli expression and C-His/Strep MMLV-RT based on the baculovirus expression system in Sf9 cells. The protein-tag strategy allowed for expression of both proteins in large amounts and application of affinity chromatography for purification without time-consuming precipitation steps. Because the protein purification procedure still includes two chromatography techniques with different chemical properties, the purity of the final product is more than 90% (FIGS. 2C and 2D). Moreover, as shown in FIG. 2E, the high yields of both purified His-Taq Pol and C-His/Strep MMLV-RT proteins from relatively small volumes of 1 L E. coli and 1 L Sf9 cultures, respectively, amassed for purified protein amounts of more than $100,000 per enzyme in market value. The quantities of Taq Pol and MMLV-RT produced by the disclosed protein expression and purification schemes at the laboratory-scale are satisfactory and do not require production upscaling to an industrial-scale facility. The disclosed protein production platform provides a simpler follow-up example for groups in research institutes with limited-resources.

Till present, there have been many research studies for enhancing and improving the enzymatic properties of Taq Pol and MMLV-RT. However, the improvements in the enzymatic performance of commercial MMLV-RTs variants are mainly to amend the efficiency of total RNA synthesis in order to generate cDNA libraries through random annealing of primers. On the other hand, commercial Taq Pol variants have enhanced rate and processivity for quicker PCR reactions and longer amplifications. In the scheme of COVID-19 detection, the RT primers are explicitly designed to be gene-specific and the RNA regions annealed by the RT primers are carefully selected to avoid erroneous interference in cDNA synthesis. These studies assume that the well-designed RT primers for cDNA synthesis conceals any possible enzymatic incompetency of the MMLV-RT and enables the detection of COVID-19 even at low threshold (see discussion below). The disclosed His-Taq Pol is not a hot-start polymerase like Platinum Taq Pol that was used as a benchmark for performance comparison during this study. However, the advantages of the hot-start PCR polymerase such as the prevention of primer-dimer formation and non-specific amplification, were not required for the TaqMan based RT-qPCR platform (FIG. 4 and Table 2). Also the use of the relatively short fragments for amplification was found to be in favor for the His-Taq Pol in the one-step RT-qPCR scheme. While not being bound by theory, the data suggests that the synergy between using TaqMan based detection system and the short size of amplicons makes the performance of the non-hot-start His-Taq Pol equally robust as the hot-start Platinum Taq Pol in the one-step RT-qPCR reaction.

The aim from the current study was to establish the one-step RT-qPCR kit with in-house enzymes. The advantages of a one-step RT-qPCR platform over a two-step process in the context of work-flow at the point-of-care diagnostics include minimal sample handling, reduced bench time, and less chances for pipetting errors and cross-contamination (FIG. 1). This makes the one-step platform the first choice for high-throughput mass screening in the diagnostic point-of-care tests. In the one-step platform, it is very crucial to choose carefully the optimal conditions for both MMLV-RT and Tag Pol to work together in the same reaction (1, 2). A potential obstacle in assembling the one-step RT-qPCR system is the inhibition of the Tag Pol's activity in the presence of MMLXV-RT. It was previously proposed that both enzymes interact with specific combination of primers (DNAs) and templates (RNAs) causing inhibitory effect (2). The present studies showed that MMLV-RT hinders the Taq Pol's activity in a portion-dependent manner; however, this inhibitory effect can be circumvented, to some extent, by increasing the amount of Taq Pol (FIG. 3C). Apart from Taq Pol and MMLV-RT ratio, sulfur-containing inorganic molecules are also known to relieve the inhibition of PCR and often added when using compositions containing two or more enzymes for the reverse transcription activity (U.S. Pat. No. 9,556,466 B2). In this study, besides adding KCl in the reaction buffer, ammonium sulfate was selected as additional salt and showed that it helped alleviate the MMLV-RT's inhibitory effect on the Taq Pol's activity (FIG. 3D). However, this alleviation disappeared when Taq Pol and MMLV-RT were pre-mixed in the absence of salts (data not shown). This result suggest that Taq Pol and MMLV-RT may directly interact with each other. However, both the SDS-PAGE (FIG. 2F) and SEC analysis (FIGS. 5A-5C) did not show any detectable physical interaction between Taq Pol and MMLV-RT, thus the cause for this adverse effect remains unclear.

Next studies verified the ideal proportions of Taq Pol and MMLV-RT in the optimal reaction buffer in the context of COVID-19 detection work-flow (FIG. 4C). Studies confirmed that 30 U: 60 ng/μL is the optimal composition for the one-step RT-qPCR platform disclosed herein, where as low as 10 RNA copies per reaction with more than 90% reproducibility was a successfully detected. The optimal amounts of MMLV-RT determined here, along with the aforementioned ratio for MMLV-RT and Taq Pol are surprisingly low given that per manufacturer's recommendations for making cDNA libraries the use of 200 units is advised, which is almost 1,000 ng of enzyme for the RT reactions with random hexamer or dT primers. The studies demonstrate that lowering the amount of MMLV-RT in the reaction is one critical parameter to make Taq Pol functional in the one-step RT-qPCR platform and that around 60 ng of MMLV-RT is sufficient to synthesize necessary cDNA for COVID-19 diagnostic purposes.

Finally, the disclosed one-step RT-qPCR system was tested with actual patient samples isolated from individuals infected by SARS-CoV2 (Table 2). 23 different patients were screened, including three who were suspected but tested negative for SARS-CoV2. All tests with the disclosed one-step RT-qPCR showed the expected true positive and negative results with a slight difference in the Ct values from Invitrogen SuperScript™ III Platinum™ One-Step RT-qPCR System. The results provide a guide on how to assemble an in-house one-step RT-qPCR kit. It is contemplated that the one-step RT-qPCR system can be successfully applied for routine SARS-CoV2 diagnostics, but more generally, it can be used to detect the presence of nucleic acids from other pathogens, in samples.

### REFERENCES

1. Chan, et al., Lancet, 2020. 395(10223): p. 514-523.
2. Lu, et al., Lancet, 2020. 395(10224): p. 565-574.
3. Wu, et al., Nature, 2020. 580(7803): p. E7-E7.
4. Lu, et al., Lancet, 2020. 395(10224): p. 565-574.
5. http://www.euro.who.int/en/health-topics/health-emergencies/coronavirus-covid-19/novel-coronavirus-2019-ncov.
6. https://www.covidvisualizer.com
7. Zhou, et al., Nature, 2020. 579(7798): p. 270-+.
8. Wu, et al., Nature, 2020. 579(7798): p. 265-+.
9. Li, et al., Science, 2005. 310(5748): p. 676-679.
10. Ge, et al., Nature, 2013. 503(7477): p. 535-+.
11. Hu, et al., Plos Pathogens, 2017. 13(11).
12. Gorbalenya, et al., Virus Research, 2006. 117(1): p. 17-37.
13. Sawicki, et al. J Virol, 2007. 81(1): p. 20-9.
14. Cui, et al., Nat Rev Microbiol, 2019. 17(3): p. 181-192.
15. Li, et al., Nature, 2003. 426(6965): p. 450-454.
16. Qian, et al., Am. J. of Resp. Cell and Mol. Biol., 2013. 48(6): p. 742-748.
17. Bosch, et al., Journal of Virology, 2003. 77(16): p. 8801-8811.
18. Li, Annual Review of Virology, Vol 3, 2016. 3: p. 237-261.
19. Wrapp, et al., Science, 2020. 367(6483): p. 1260-+.
20. Walls, et al., PNAS 2017. 114(42): p. 11157-11162.
21. Gui, et al., Cell Research, 2017. 27(1): p. 119-129.
22. Pallesen, et al., PNAS, 2017. 114(35): p. E7348-E7357.
23. Yuan, et al., Nature Communications, 2017. 8.
24. de Haan, et al., Journal of Virology, 1998. 72(8): p. 6838-6850.
25. Woo, et al., Analysis. Viruses-Basel, 2010. 2(8): p. 1804-1820.
26. Nieto-Torres, et al., Viruses-Basel, 2015. 7(7): p. 3552-3573.
27. Schoeman, et al, Virology Journal, 2019. 16.
28. Fung, et al, Annual Rev. of Microbiol., Vol 73, 2019. 73: p. 529-557.
29. Luk, et al., Infection Genetics and Evolution, 2019. 71: p. 21-30.
30. Neuman, et al., J Struct Biol, 2011. 174(1): p. 11-22.
31. Schoeman, et al, Virol J, 2019. 16(1): p. 69.
32. Guan, et al., Science, 2003. 302(5643): p. 276-278.
33. Alagaili, et al., Mbio, 2014. 5(2).
34. Al-Ahmadi, et alZoonoses and Public Health, 2020. 67(4): p. 382-390.
35. http://www.undp.org/content/undp/en/home/coronavirus/socio-economic-impact-of-covid-19.html.
36. Niemz, et al. Trends Biotechnol, 2011. 29(5): p. 240-50.
37. Dohla, et al., Public Health, 2020. 182: p. 170-172.
38. Yan, et al., Clin Microbiol Infect, 2020. 26(6): p. 773-779.
39. Yu, et al., Clin Chem, 2020.
40. Park, et al . . . J Mol Diagn, 2020. 22(6): p. 729-735.
41. Kellner, et al., Nat Protoc, 2019. 14(10): p. 2986-3012.
42. Broughton, et al., Nat Biotechnol, 2020.
43. Zahir Ali, R. A., Ahmed MahasI, Gundra Sivakrishna Rao, Muhammad Tehseen, Tin Marsic, Rahul Salunke, Amit K. Subudhi, Sharif M. Hala, Samir M. Hamdan, Arnab Pain, Norhan Hassan, and Magdy M. Mahfouz., iSCAN: An RT-LAMP-coupled CRISPR-Cas12 module for rapid, sensitive detection of SARS-CoV-2. medRxiv, 2020.
44. Corman, et al., Euro Surveill, 2020. 25(3).
45. http://www.cdc.gov/coronavirus/2019-ncov/lab/rt-pcr-panel-primer-probes.html.
46. Verma, J Virol, 1975. 15(4): p. 843-54.
47. Gerard, et al. J Virol, 1975. 15(4): p. 785-97.
48. Roth, M. J., N. Tanese, and S. P. Goff, Purification and characterization of murine retroviral reverse transcriptase expressed in Escherichia coli. J Biol Chem, 1985. 260(16): p. 9326-35.
49. Goff, et al. J. of Cell Comm. and Signaling, 2008. 2(1-2): p. 25-45.
51. Sellner, et al. Nucleic Acids Research, 1992. 20(7): p. 1487-1490.
52. Yano, et al., J. of Asia-Pacific Entomology, 2019. 22(2): p. 453-457.
53. Engelke, et al., Analytical Biochemistry, 1990. 191(2): p. 396-400.
54. Baltimore, et al. Nature, 1970. 226(5252): p. 1209-11.
55. Chien, et al. J Bacteriol, 1976. 127(3): p. 1550-7.

1. An RT-PCR reagent composition comprising a Moloney Murine Leukemia Virus Reverse Transcriptase (MMLV-RT) and a Thermus aquaticus DNA polymerase (Taq Pol) in effective amounts and in a buffer effective for one-step RT-PCR.

2. The composition of claim 1 comprising MMLV-RT and Taq Pol in a 2:1 ratio.

3. The composition of claim 2, wherein the ratio comprises nanograms of MMLV-RT to units (U) of Taq Pol.

4. The composition of claim 1 comprising from about 40 ng to about 80 ng MMLV-RT, from about 20 U to about 40 U Taq Pol, or combinations thereof.

5. The composition of claim 4 comprising about 60 ng MMLV-RT and about 30 U Taq Pol.

6. The composition of claim 1, wherein; (a) the MMLV-RT comprises the amino acid sequence of SEQ ID NO:1 or an amino acid sequence comprising at least 85% sequence identity to SEQ ID NO:1, (b) the Taq Pol comprises the amino acid sequence of SEQ ID NO:2 or an amino acid sequence comprising at least 85% sequence identity to SEQ ID NO:2; and/or (c) the MMLV-RT and/or Taq Pol further comprise one or more tag sequences at the N-terminus and/or C-terminus.

7. (canceled)

8. (canceled)

9. The composition of claim 8, where the tag sequence is selected from His tag and Strep tag.

10. The composition claim 1, wherein the buffer comprises one or more of a salts a reducing agent, a chelating agent, a detergent a buffering agent, a plurality of deoxynucleoside triphosphates (dNTPs), or combinations thereof.

11. The composition of claim 10, wherein the one or more salts are selected from the group comprising KCl, MgCl2, and (NH4)2SO4.

12. The composition of claim 10, wherein the dNTPs are selected from the group consisting of dATP, dTTP, dGTP, and dCTP.

13. The composition of claim 10 comprising 20-50 mM Tris-HCl (pH 8.5); 75-150 mM KCl; 2-4 mM MgCl₂; 0.5-2 mM DTT; 200-500 μM dNTPs and 13.5 mM (NH4)2SO4.

14. A method of performing a one-step RT-PCR comprising forming a mixture by combining an RNA sample/template and a plurality of primers with the composition of claim 1 and incubating the mixture under conditions sufficient to amplify one or more DNA molecules complementary to one or more portions of the RNA sample/template.

15. A method of detecting presence of a nucleic acid in a sample comprising combining the sample and plurality of primers specific to the nucleic acid with the composition of claim 1 under conditions sufficient for amplification of the nucleic acid, and detecting the nucleic acid amplification product, thereby detecting presence of the nucleic acid in the sample.

16. The method of claim 15, wherein the nucleic acid is derived from a coronavirus, preferably a severe acute respiratory syndrome-related coronavirus, more preferably SARS-CoV-2.

17. The method of claim 14, wherein the sample is an RNA sample derived from mucus, sputum (processed or unprocessed), saliva, bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.

18. A method of diagnosing a subject for infection with a virus comprising detecting the presence of a viral nucleic acid in a sample from the subject by the method of claim 15, wherein detecting the amplification product indicates the subjects is infected with the virus.

19. The method of claim 18, wherein the subject exhibits or does not exhibit symptoms of a disease, disorder, or condition associated with the virus, and/or the method further comprising treating the subject, wherein the subject was diagnosed as infected with the virus.

20. (canceled)

21. The method of claim 18, wherein the subject is human.

22. The composition of claim 1 wherein the MMLV-RT sequence comprises a sequence represented by SSAG-A-L1-B-L2-C, where: (i) SSAG is an amino acid sequence or a variant thereof made by conservative substitution of the amino acids therein; (ii) A is a peptide cleave sequence, (iii) B is a protein tag as disclosed herein, (iv) L1 and L2 are optional first linkers, and (v) B is a second protein tag.

23. The composition of claim 1, wherein the MMLV-RT comprises SSAGENLYFQGSSSHHHHHHHHGGGSAWSHPQFEK (SEQ ID NO: 20).
